(19) United States
(12) Patent Application Publication
(10) Pub. No.: US 2025/0152565 A1
(43) Pub. Date: May 15, 2025

(54) EFFECTS OF LYSERGIC ACID DIETHYLAMIDE (LSD) AND OF LSD ANALOGS TO ASSIST PSYCHOTHERAPY FOR GENERALIZED ANXIETY DISORDER OR OTHER ANXIETY NOT RELATED TO LIFE-THREATENING ILLNESS

(71) Applicant: UNIVERSITÄTSSPITAL BASEL, Basel (CH)

(72) Inventors: Matthias Emanuel LIECHTI, Oberwil (CH); Friederike Sophie HOLZE, Naters (CH)

(21) Appl. No.: 19/020,135

(22) Filed: Jan. 14, 2025

Related U.S. Application Data
(63) Continuation of application No. 17/672,670, filed on Feb. 15, 2022, now abandoned.
(60) Provisional application No. 63/151,611, filed on Feb. 19, 2021.

(51) Int. Cl. A61K 31/437; A61K 45/06; A61P 25/22

(52) U.S. Cl. CPC A61K 31/437; A61K 45/06; A61P 25/22

(57) ABSTRACT

A method of treating anxiety disorders specifically not associated with causes such as a life-threatening serious somatic illness, by administering a psychedelic to an individual, and treating anxiety and specifically causing reductions in the rating scale score measures of anxiety (STAI global or state or trait anxiety) and/or measures of depression (HDRS or BDI scores) and/or measures of general psychological distress (SCL-90 ratings) in the patient for several weeks beyond administration of the psychedelic. A method of treating anxiety, by administering a psychedelic (preferably LSD) to an individual having anxiety not associated with causes such as a life-threatening serious somatic illness, and inducing positive acute drug effects and positive long-term therapeutic effects in the individual.

### BACKGROUND OF THE INVENTION

### 1. Technical Field

The present invention relates to the use of LSD and LSD analogs or derivatives to induce a psychedelic state and assisting psychotherapy for generalized anxiety disorder.

### 2. Background Art

Psychedelics including lysergic acid diethylamide (LSD) are substances capable of inducing unique subjective effects including alterations of consciousness, positive emotions, enhanced introspection, changes in the perception of the environment, the body, and the self as well as synesthesia, mystical-type experiences, and experiences of ego dissolution (Carhart-Harris et al., 2016b; Dolder et al., 2016; Holze et al., 2021; Liechti, 2017; Passie et al., 2008; Schmid et al., 2015).

All serotonergic psychedelics including LSD, psilocybin, DMT, and mescaline are agonists at the 5-HT_{2A} receptor (Rickli et al., 2016) and may therefore produce overall largely similar effects. Additionally, psychedelic substances produce their acute effects in humans via activation of the serotonin 5-HT_{2A} receptor as specifically shown in clinical studies for LSD (Holze et al., 2021; Preller et al., 2017).

Acute effects of psychedelics that may contribute to their therapeutic benefits include enhancing the therapeutic relationship by increased openness, trust, feelings of connectedness or emulsion with people, insight in psychological problems and stimulation of neuroregenerative processes as described in detail elsewhere (Vollenweider et al., 2020).

Psychedelics have been investigated as potential treatments of medical conditions. Clinical trials documented beneficial effects in using LSD in patients with addiction (Krebs et al., 2012), in patients with anxiety specifically associated with life-threatening illness (Gasser et al., 2014; Gasser et al., 2015), and in using psilocybin in patients with major depression (Carhart-Harris et al., 2016a; Davis et al., 2021; Griffiths et al., 2016; Roseman et al., 2017; Ross et al., 2016), anxiety associated with terminal illness (Griffiths et al., 2016: Grob et al., 2011; Ross et al., 2016), and in different forms of addiction (Bogenschutz, 2013; Bogenschutz et al., 2015; Garcia-Romeu et al., 2019; Garcia-Romeu et al., 2015; Johnson et al., 2014; Johnson et al., 2016). Additionally, there is limited evidence that the psychedelic brew Ayahuasca, which contains the active psychedelic substance N,N-dimethyltryptamine (DMT) (Dominguez-Clave et al., 2016) may alleviate depression (Dos Santos et al., 2016; Palhano-Fontes et al., 2019; Sanches et al., 2016).

Several studies have included patients with anxiety in trials with psychedelics. Several older studies were conducted in the 1950-70s using psychedelics in patients with existential anxiety in association with advanced-stage cancer and/or dying (Grof et al., 1973; Kast. 1966; Kast et al., 1964; Pahnke et al., 1969).

Grob et al. conducted the first modem double-blind, placebo (niacin)-controlled study using psilocybin in twelve patients with advanced-stage cancer and anxiety (Grob et al., 2011). This study established primarily the feasibility and safety of administering moderate doses of psilocybin to patients with cancer and anxiety. Some of the data revealed a positive trend toward improved mood and anxiety. Specifically, there was a trend effect of psilocybin on mood on day 1 and 2 weeks after drug administration but no significant improvement of anxiety (FIG. 1). The results supported the need for further research. Psilocybin acutely induced a psychedelic state assessed with the 5-Dimension of Altered States of Consciousness scale (5D-ASC; FIG. 2) also used in the present invention (Grob et al., 2011). The diagnosis of anxiety varied in this study and included acute stress disorder, generalize anxiety disorder, anxiety disorder due to cancer, or adjustment disorder with anxiety, but importantly, advanced stages of cancer were present in all patients.

Griffiths et al. conducted a randomized placebo-controlled, double-blind trail in patients with depression and anxiety in 51 patients with life-threatening cancer. Depressed mood was defined as meeting criteria for major depressive disorder, dysthymic disorder, or adjustment disorder with anxiety and depressed mood. Anxiety was defined as meeting criteria for generalized anxiety disorder, adjustment disorder with anxiety, chronic, or adjustment disorder with anxiety and depressed mood. All 51 participants had a potentially life-threatening cancer diagnosis, with 65% having recurrent metastatic disease. The study used a cross-over design with administration of a very low (placebo-like) dose of 1 or 3 mg/70 kg psilocybin versus a high dose of 22 or 30 mg/70 kg of psilocybin and 5 weeks between sessions and a 6-month follow-up. High-dose psilocybin produced large decreases in depressed mood and anxiety, along with increases in quality of life and decreases in death anxiety (FIGS. 3A-3C). At 6-month follow-up, these changes were sustained (FIGS. 3A-3C), with about 80% of participants continuing to show clinically significant decreases in depressed mood and anxiety. Participants attributed improvements in attitudes about life/self, mood, relationships, and spirituality to the high-dose experience, with >80% endorsing moderately or greater increased well-being/life satisfaction (Griffiths et al., 2016). Higher acute Mystical Experience Questionnaire (MEQ) scores in response to psilocybin were positively associated with lower anxiety and depression 5 weeks after psilocybin administration (FIGS. 4A-4B) indicating that greater positive acute psilocybin effects are related to better long-term therapeutic benefits (Griffiths et al., 2016).

Ross et al. conducted a randomized controlled trial of psilocybin treatment for anxiety and depression in 29 patients with life-threatening cancer (Ross et al., 2016). 62% of the patients had advanced cancers stage III or IV. 26 patients had adjustment disorder and only three patients had generalized anxiety disorder. Patients were randomly assigned to single-dose psilocybin (0.3 mg/kg) or niacin placebo. The primary outcomes were anxiety and depression assessed between groups prior to the crossover at 7 weeks. Psilocybin produced improvements in anxiety and depression and increased quality of life (FIGS. 5A-5C and 6A-6C). Again greater mystical-type experiences were associated with better treatment outcomes long-term.

Carhart-Harris et al. conducted an open-label feasibility study of the effects of psilocybin (10 mg and 25 mg 7 days apart) with psychological support for the treatment of treatment-resistant depression in 12 patients (Carhart-Harris et al., 2016a). There was no control group. Psilocybin was well-tolerated by all of the patients. The adverse reactions noted were transient anxiety during drug onset (all patients), transient confusion or thought disorder (nine patients), mild and transient nausea (four patients), and transient headache (four patients). Relative to baseline, depressive symptoms were markedly reduced 1 week (p=0.002) and 3 months (p=0.003) after high-dose treatment (FIG. 7). Marked and sustained improvements in anxiety and anhedonia were also noted. This study provided preliminary support for the safety and efficacy of psilocybin for treatment-resistant depression (Carhart-Harris et al., 2016a).

Davis et al. conducted a randomized trial in 27 patients with major depression who were randomized to two psilocybin sessions (session 1: 20 mg/70 kg; session 2: 30 mg/70 kg one week later) or a waiting list for delayed treatment (Davis et al., 2021). The primary outcome was depression severity (Hamilton Depression Rating Scale, HAMD) at 1 and 4 weeks after session 2. 24 patients completed the intervention. The mean (SD) HAMD scores at weeks 1 and 4 (8.0 [7.1] and 8.5 [5.7]) in the immediate treatment group were statistically significantly lower than the scores at the comparable time points of weeks 5 and 8 (23.8 [5.4] and 23.5 [6.0]) in the delayed treatment group (FIG. 8). The effect sizes were large at week 5 (Cohen d=2.2; 95% CI, 1.4-3.0; P<0.001) and week 8 (Cohen d=2.6; 95% CI, 1.7-3.6; P<0.001). The findings suggest that psilocybin with therapy is efficacious in treating depression, thus extending the results of previous studies of this intervention in patients with cancer and depression and of a nonrandomized study in patients with treatment-resistant depression (Davis et al., 2021).

Gasser et al. conducted the first modem study with LSD in patients with life-threatening illness and related anxiety (Gasser et al., 2014). Treatment included drug-free psychotherapy sessions and two LSD sessions 2 to 3 weeks apart. Twelve patients were included and eight received 200 μg of LSD (per os, as capsules) twice in two sessions 2-3 weeks apart and three patients received placebo (a low dose of LSD of 20 μg). All patients had an increased level of anxiety (>40 points) on either the state or trait scale of the State-Trait Anxiety Inventory (STAI) and half were also diagnosed with generalized anxiety disorder according to the Diagnostic and Statistical Manual (DSM)-IV. The study documented a significant decrease in STAI anxiety 2 months after the two LSD sessions compared with baseline anxiety scores (FIGS. 9A-9B). In contrast, STAI scores did not decrease after the placebo sessions. However, the placebo control group was too small for statistical comparison with the treatment group. The study also documented sustained decreases in anxiety up to 12 months after the LSD treatments, but a placebo group was missing (Gasser et al., 2015). There were no drug-related severe adverse effects, no panic reactions, or other medical or psychiatric complications. The study authors concluded that LSD can safely be used in patients with anxiety disorder but that larger controlled studies are warranted to confirm efficacy (Gasser et al., 2014). A follow-up study at 12 months also documented no adverse effects up to 12 months and provided a qualitative account of the beneficial effects of LSD in this study (Gasser et al., 2015).

LSD was also used in a few smaller older studies in patients with depression without somatic illness (Savage, 1952). LSD was also used experimentally and temporarily in the absence of supporting study data in patients with affective disorders (depression, anxiety, obsessive compulsive disorder) in Switzerland from 1988-1993 and outside clinical studies (Gasser, 1996). There was similar use of LSD in the 1960-70s outside placebo-controlled clinical studies (Pahnke et al., 1970). However, clinical study data on the efficacy of LSD in patients with anxiety without somatic illness is lacking. Even for other psychedelics such as psilocybin, there is only data on anxiety and depression associated with cancer (Griffiths et al., 2016; Ross et al., 2016) or on depression (Carhart-Harris et al., 2018; Carhart-Harris et al., 2016a; Davis et al., 2021) but not for anxiety disorders not linked to somatic disease.

Schmid et al. described the regulated therapeutic use of LSD in psychiatric patients in Switzerland. The observational study described 11 patients treated with LSD in addition to psychotherapy. Patients suffered from post-traumatic stress disorder (4), major depression (2), anxious personality disorder (1), narcissistic personality disorder (1), obsessive compulsive disorder (1), and dissociative disorder (2). LSD produced acute effects on the 5D-ASC scale and MEQ that were similar to those describe in healthy subjects (FIGS. 10 and 11) and those reported in other studies using psilocybin in patients with anxiety associated with cancer or patients with depression (Grob et al., 2011: Roseman et al., 2017) or in a study using LSD to treat anxiety associated with cancer (Gasser et al., 2014; Liechti et al., 2017).

After no official research in humans in the last 40 years, LSD is currently being investigated in healthy subjects. Carhart-Harris and colleagues performed an experimental single-blind, within-subject, placebo-controlled pilot study in 10 healthy volunteers using 40-80 μg LSD administered intravenously (Carhart-Harris et al., 2015; Kaelen et al., 2015). LSD produces subjective effects including “wandering thoughts”, “feeling amazing”, and “feeling inner warmth” (Kaelen et al., 2015). LSD slightly elevated blood pressure and heart rate (Kaelen et al., 2015). LSD enhanced suggestibility (Carhart-Harris et al., 2015), enhanced the emotional response to music (Kaelen et al., 2015), and induced synaesthesia-like experiences (Terhune et al., 2016). The same group then conducted another larger placebo controlled crossover study in 20 subjects using a dose of 75 μg LSD (intravenously, corresponding to about 100 μg oral LSD) and included a functional magnetic resonance imaging (fMRI) scanning session (Carhart-Harris et al., 2016b; Carhart-Harris et al., 2016c; Kaelen et al., 2016; Lebedev et al., 2016; Roseman et al., 2016: Speth et al., 2016; Tagliazucchi et al., 2016). All participants had at least one previous experience with a classic psychedelic substance and on average used LSD 14 times (Carhart-Harris et al., 2016b). LSD produced heightened mood, a blissful state, and also acute psychosis-like symptoms including thought disorder, delusional thinking and paranoia. There was only a small increase in anxiety, significantly smaller than the blissful experience. Overall, there was a bias towards positive affect. As previously described for psilocybin (Griffiths et al., 2008; MacLean et al., 2011), LSD produced lasting effects (Carhart-Harris et al., 2016b). LSD increased optimism and trait openness at two weeks compared with placebo. At two weeks, there was no effect on delusional thinking and a trend towards less distress and less preoccupation with delusional thoughts (Carhart-Harris et al., 2016b). The data indicates that in healthy subjects, psychedelics increased openness and psychological wellbeing mid- to long-term (Carhart-Harris et al., 2016b; Griffiths et al., 2008; MacLean et al., 2011). Consistently, there is no evidence of psychological or psychiatric problems associated with the use of psychedelic substances by healthy subjects according to follow-up data from placebo-controlled studies (Carhart-Harris et al., 2016b; Studerus et al., 2011) or epidemiological data (Johansen et al., 2015; Krebs et al., 2013b).

Several double-blind, placebo-controlled, random order cross-over phase I studies were conducted in healthy subjects in Switzerland. The first study used a dose of 200 μg oral LSD in 16 subjects (8 male, 8 female) and characterized the psychological, physiological, endocrine and pharmacokinetic effects of LSD (Dolder et al., 2015b; Schmid et al., 2015; Strajhar et al., 2016). Administration of LSD produced pronounced alterations in waking consciousness that lasted 12 hours. The predominant effects induced by LSD included visual hallucinations, audiovisual synesthesia, and positively experienced derealization and depersonalization phenomena. Subjective well-being, happiness, closeness to others, openness, and trust were increased by LSD. LSD significantly increased blood pressure, heart rate, body temperature, pupil size, plasma cortisol, prolactin, oxytocin, and epinephrine. Adverse effects produced by LSD completely subsided within 72 hours. No severe acute adverse effects were observed (Schmid et al., 2015; Strajhar et al., 2016). Maximal concentrations of LSD were reached 1.5 hours after administration. Concentrations then decreased following first-order kinetics with a half-life of 3.6 hours up to 12 hours and slower elimination thereafter. No sex differences were observed in the pharmacokinetic profiles of LSD. The acute subjective and sympathomimetic responses to LSD were closely associated with the concentrations in plasma over time and exhibited no acute tolerance (Dolder et al., 2015b).

The second study tested effects of a dose of LSD of 100 μg in 24 healthy subjects (12 male, 12 female) (Dolder et al., 2016). A next study tested effects of doses of 25, 50, 100, 200 μg and placebo in 16 healthy subjects (Holze et al., 2021). Another study compared effects of 100 μg LSD with those of MDMA and d-amphetamine (Holze et al., 2020b).

In an fMRI study, it was found that LSD decreased amygdala reactivity to fearful stimuli. The result stands in line with findings obtained after administration of psilocybin, where attenuated recognition of negative facial expressions (Kometer et al., 2012; Schmidt et al., 2013) and reduced amygdala BOLD response to fearful faces (Kraehenmann et al., 2015) were reported. Additionally, the amygdala deactivation by LSD was associated with its acute subjective psychedelic effects. Decreasing amygdala reactivity using psychedelic substances thus reflects a potentially therapeutic effect and might reduce perception of negative emotions and facilitate the therapeutic alliance.

LSD also positively altered the processing of emotional information by decreasing the recognition of fearful and sad faces and enhancing emotional empathy and prosociality (Dolder et al., 2016) similar to MDMA (Hysek et al., 2014). These effects of LSD in healthy participants likely have translational relevance to LSD-assisted psychotherapy in patients and can be expected to reduce the perception of negative emotions and facilitate the therapeutic alliance.

LSD has been investigated in patients with life-threatening diseases such as cancer. The above described studies did not include patients with generalized anxiety disorder without a somatic disease and it is therefore unknown and unexamined whether patients with anxiety in the absence of somatic illness would benefit from psychedelic-assisted therapy. While there is evidence that psilocybin and LSD improve anxiety, depression, and quality of life in patients with anxiety linked to cancer, it cannot be assumed that patients with generalized anxiety or social anxiety would also benefit.

Generalized anxiety disorder and other types of anxiety such as social anxiety disorder are highly prevalent and represent a quantitatively larger health problem and cause higher costs to society than adjustment disorder to cancer and other life-threatening diseases. Additionally, pharmacological treatment options are limited including the chronic administration of medications such as serotonin transporter inhibitors (citalopram, paroxetine), quetiapine, or pregabalin. These medications have substantial adverse drug effects and are of limited efficacy. Psychotherapy can effectively be used. However, additional and supplementary treatment options are needed.

Therefore, there remains a need for an effective treatment for generalized anxiety disorder.

### SUMMARY OF THE INVENTION

The present invention provides for a method of treating anxiety disorders specifically not associated with causes such as a life-threatening serious somatic illness, by administering a psychedelic to an individual, and treating anxiety and specifically causing reductions in the rating scale score measures of anxiety (STAI global or state or trait anxiety) and/or measures of depression (HDRS or BDI scores) and/or measures of general psychological distress (SCL-90 ratings) in the patient for several weeks beyond administration of the psychedelic.

The present invention provides for a method of treating anxiety, by administering a psychedelic (preferably LSD) to an individual having anxiety not associated with causes such as a life-threatening serious somatic illness, and inducing positive acute drug effects and positive long-term therapeutic effects in the individual.

### DESCRIPTION OF THE DRAWINGS

Other advantages of the present invention are readily appreciated as the same becomes better understood by reference to the following detailed description when considered in connection with the accompanying drawings wherein:

FIGS. 1A-1B are graphs of the effects of psilocybin on depression in patients with advanced-stage cancer and anxiety in the prior art, FIG. 1A shows BDI Score, and FIG. 1B shows STAI State Anxiety Score;

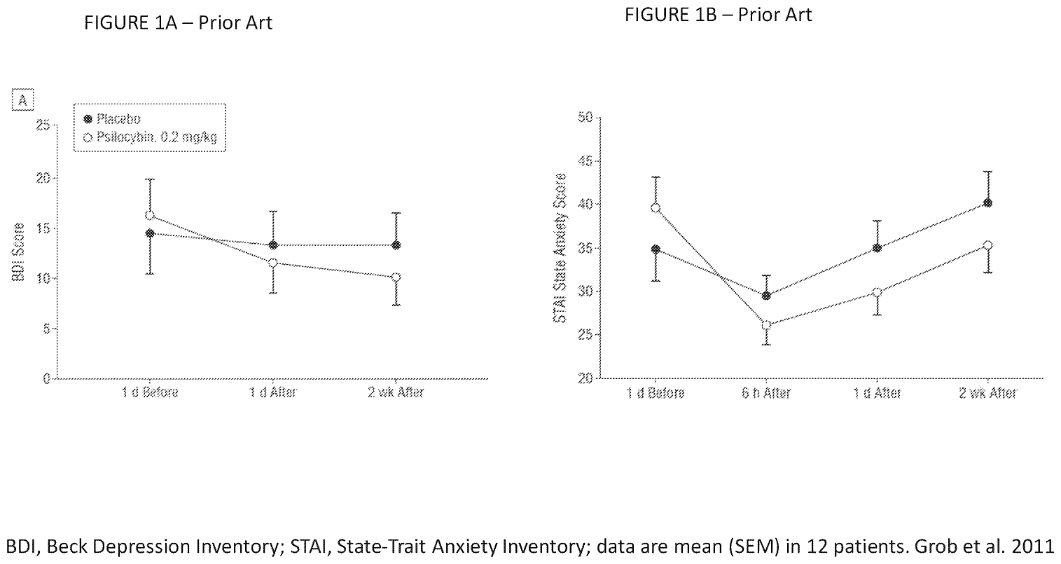

FIGS. 2A-2B are graphs showing acute alterations in mind produced of psilocybin in patients with advanced-stage cancer and anxiety in the prior art, FIG. 2A shows 5D-ASC Dimension, and FIG. 2B shows additional alterations;

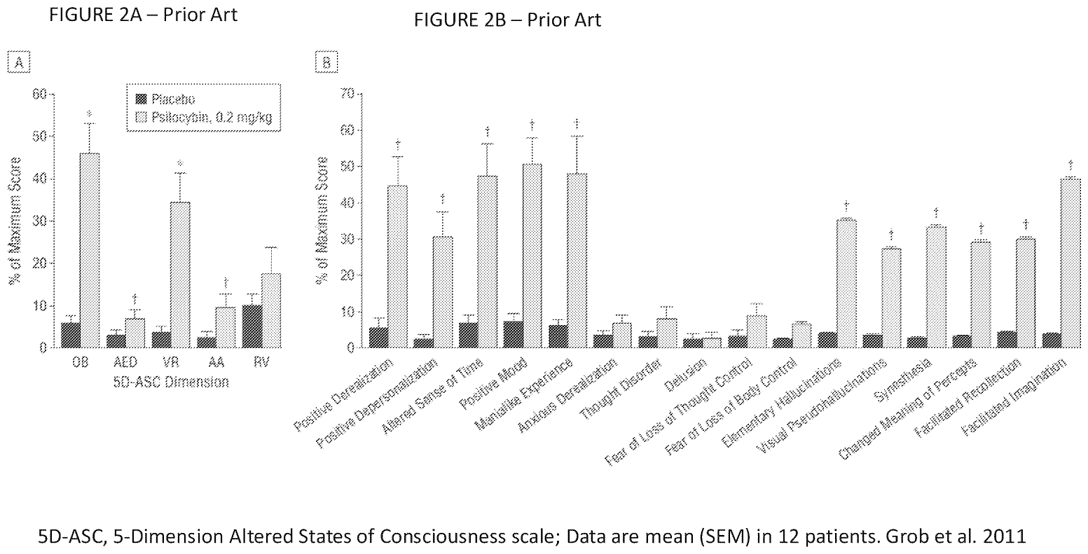

FIGS. 3A-3C are graphs showing reductions in depression (FIG. 3A) and anxiety (FIG. 3B) and increases in quality of life (FIG. 3C) in cancer patients treated with psilocybin in the prior art;

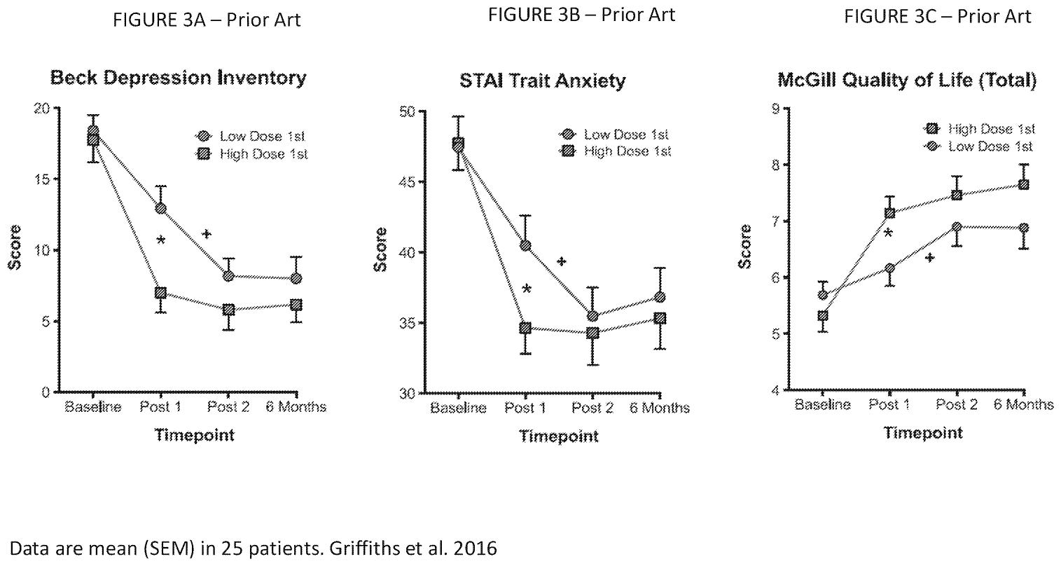

FIGS. 4A-4B are graphs showing the association of the psilocybin-induced acute mystical experiences with the change in anxiety (FIG. 4A) and depression (FIG. 4B) five weeks after the psilocybin administration in patients with cancer pre-crossover in the prior art;

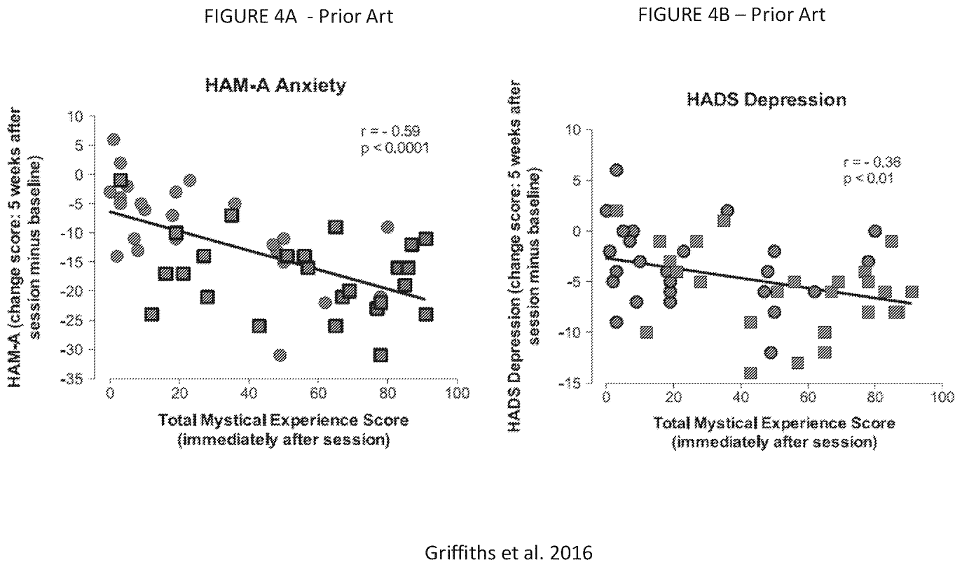

FIGS. 5A-5C are graphs showing reductions in depression (FIG. 5A), state-anxiety (FIG. 5B) and trait-anxiety (FIG. 5C) after a single dose of psilocybin and compared with niacin placebo in patients with cancer in the prior art;

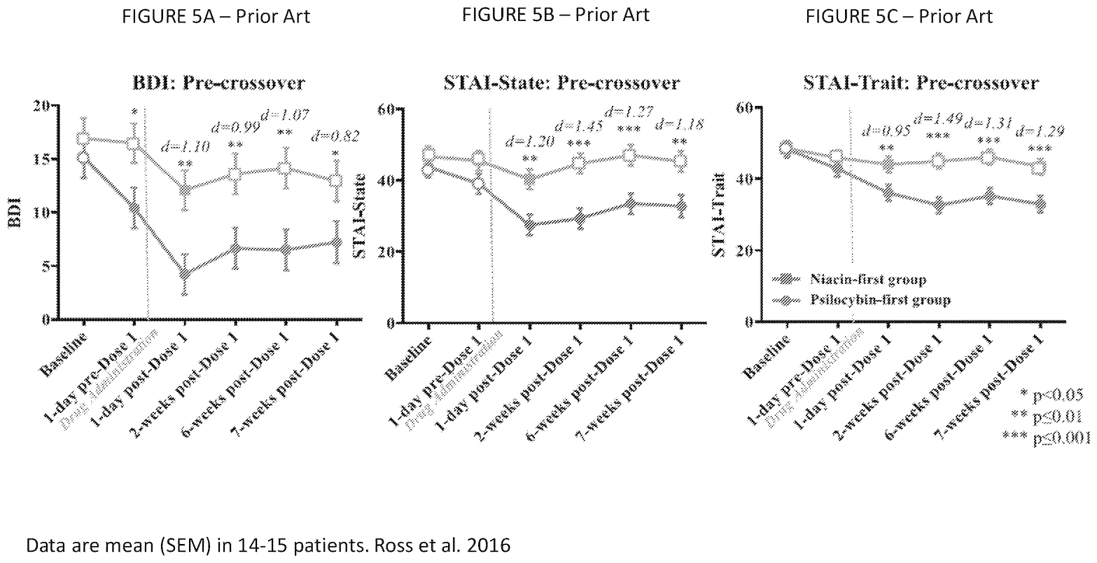

FIGS. 6A-6C are graphs showing reductions in depression (FIG. 6A), state-anxiety (FIG. 6B) and trait-anxiety (FIG. 6C) after a single dose of psilocybin and compared with niacin placebo in patients with cancer post-crossover in the prior art;

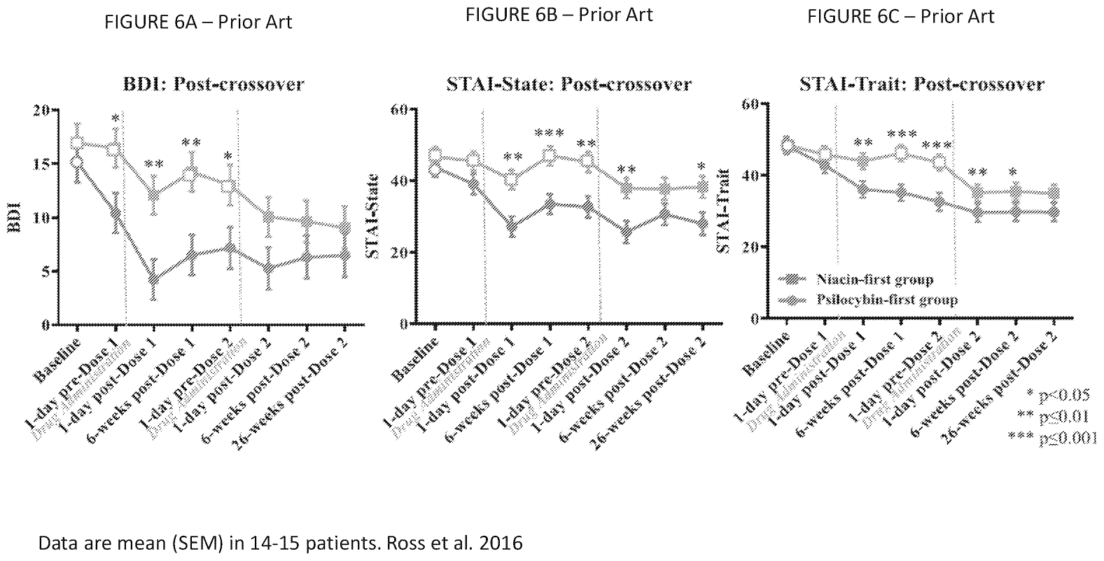

FIG. 7 shows effects of a dose of psilocybin in patients with treatment-resistant depression on depression scores 1 week and three months after administration in an open-label study without control group in the prior art;

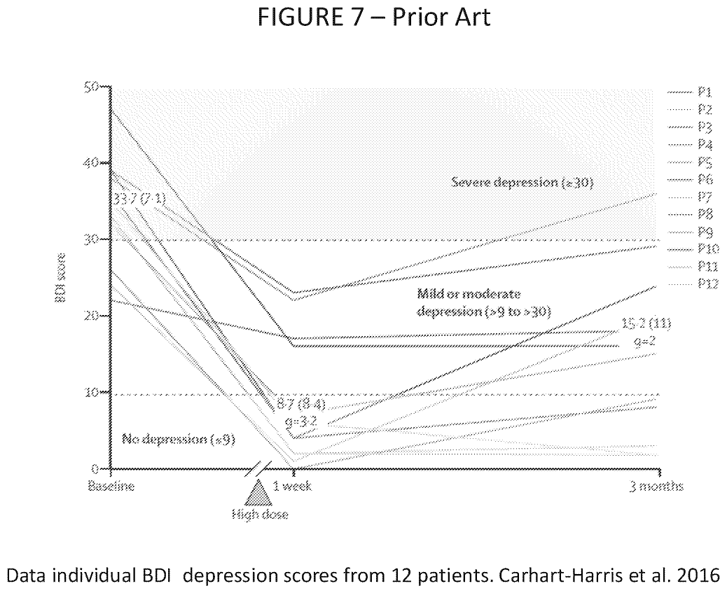

FIG. 8 shows effects of two doses of psilocybin in patients with major depression on depression scores and compared with patients waiting for later treatment in the prior art;

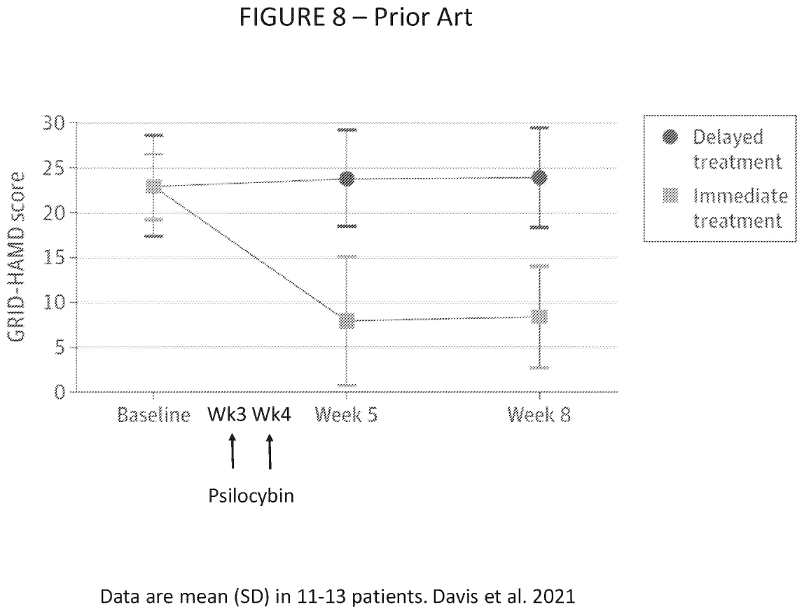

FIGS. 9A-9B are graphs showing effects of two LSD sessions on state (FIG. 9A) and trait (FIG. 9B) anxiety scores in the LSD (n=8) and placebo (n=3) group two months after drug administration in patients with life-threatening illness, placebo-treated patients then crossed over to treatment and data is shown 2 months after and for all remaining patients from both group after 12 months (n=9) in the prior art;

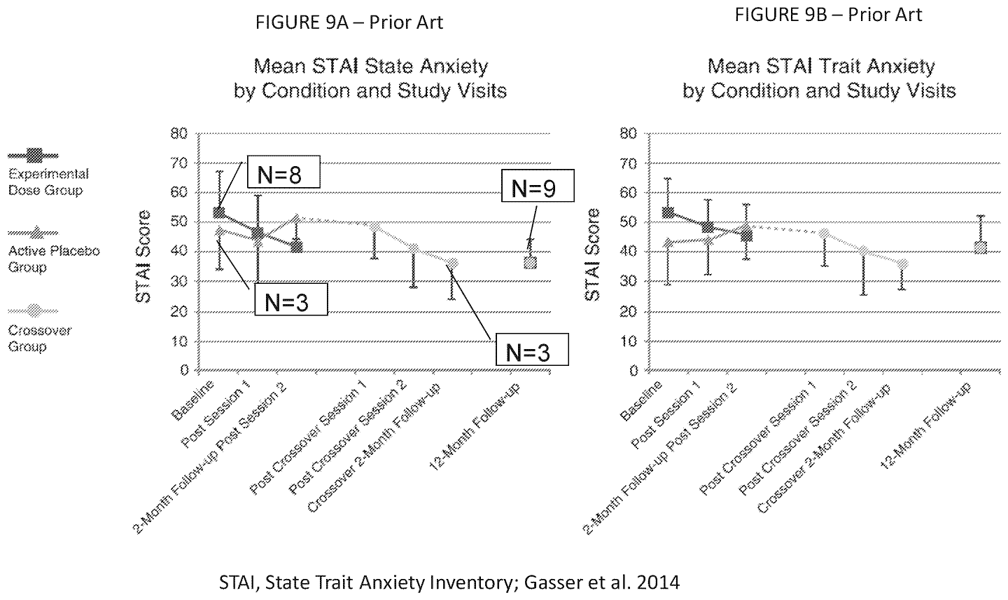

FIG. 10 is a graph showing acute alterations of the mind induced with LSD in psychiatric patients and healthy study subjects in the prior art;

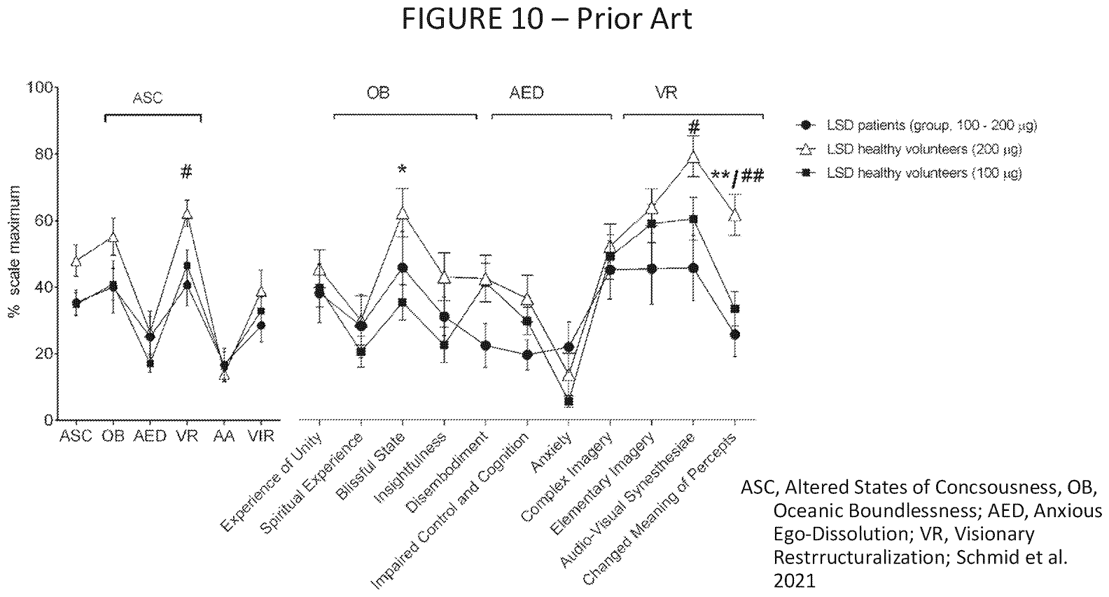

FIG. 11 shows mystical-type experiences in patients with psychiatric patients and healthy subjects in the prior art;

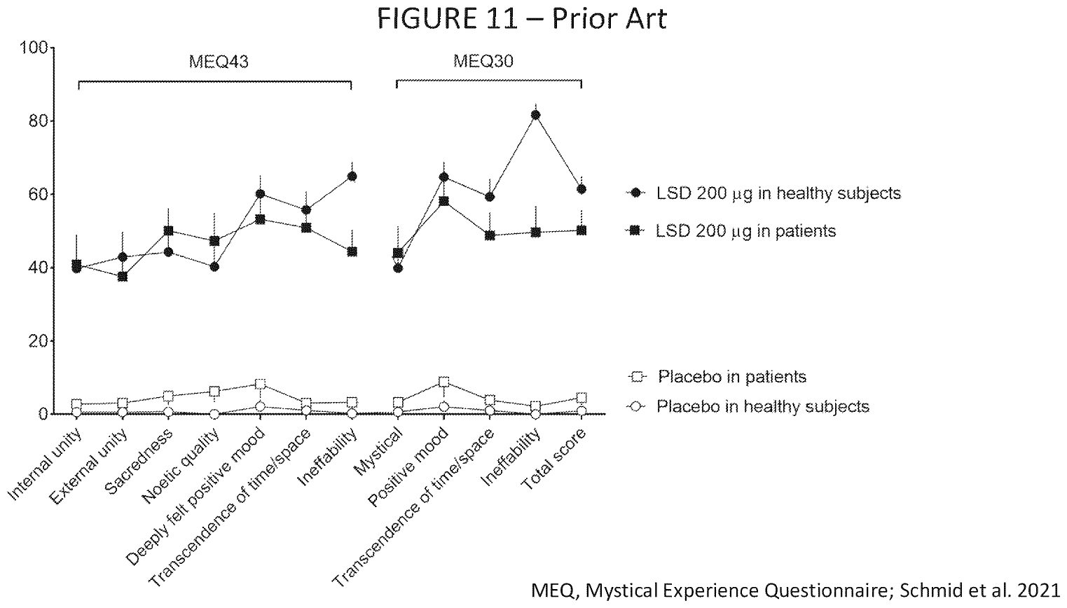

FIG. 12 is a schematic of the patient enrolment plan of the study (example) in the present invention;

FIG. 13 is a representation of the study visit plan including the outcome measurements;

FIG. 14 is a table showing the patient characteristic for the patients with anxiety disorder treated in the example study;

FIGS. 15A-15F are graphs of the effects of LSD and placebo on anxiety, depression, and psychological distress in patients with anxiety disorder between subjects (n=9-10/group), FIG. 15A shows STAI-S, FIG. 15B shows STAI-T, FIG. 15C shows STAI-G, FIG. 15D shows HDRS, FIG. 15E shows BDI, and FIG. 15F shows SCL-90 Global;

FIGS. 16A-16F are graphs of the effects of LSD versus placebo on anxiety and depression and psychological distress in patients with anxiety disorder within-subjects (n=19 per group), FIG. 16A shows STAI-S, FIG. 16B shows STAI-T, FIG. 16C shows STAI-G, FIG. 16D shows HDRS, FIG. 16E shows BDI, and FIG. 16F shows SCL-90 Global;

FIG. 17 is a graph showing acute alterations of the mind induced by LSD or placebo;

FIG. 18 is a graph showing acute mystical-type effects induced by LSD or placebo;

FIG. 19 is a table listing the correlations coefficients between the acute effects of LSD and the therapeutic effects of LSD at 2 weeks after the second dose; and

FIG. 20 shows a list of adverse events listed as total number of reports at all visits.

### DETAILED DESCRIPTION OF THE INVENTION

The present invention provides for a method of treating anxiety not associated with a life-threatening somatic illness, such as generalized anxiety disorder, by administering a psychedelic (preferably LSD) to an individual, and treating anxiety, preferably by specifically causing reductions in the rating scale score measures of anxiety (STAI global or state or trait anxiety), measures of depression (HDRS or BDI scores), and/or measures of general psychological distress (SCL-90 ratings) in the individual for several weeks beyond administration of the psychedelic.

“Generalized anxiety disorder” as used herein refers to a condition that is characterized by persistent and excessive worry. Generalized anxiety disorder is present when an individual has difficulty controlling worry on more days than not for at least six months and has at least three defined symptoms (such as feeling nervous, irritable, or on edge, having a sense of impending danger, panic, or doom, having an increased heart rate, breathing rapidly (hyperventilation), sweating, and/or trembling, feeling weak or tired, difficulty concentrating, having trouble sleeping, and experiencing gastrointestinal (GI) problems). Generalized anxiety disorder is different from anxiety brought on by a specific stressor, such as illness.

The individuals treated herein can suffer from generalized anxiety disorder, as well as other anxiety disorders, such as social anxiety disorder (social phobia, where everyday interactions cause anxiety, fear, self-consciousness, and embarrassment), panic disorder (unexpected panic attacks causing sudden, overwhelming terror for no reason and racing heart, breathing difficulties, and sweating), or other phobias, adjustment disorders, or post-traumatic stress disorder. The method can also treat depression or low mood associated or co-present with anxiety.

The present invention preferably uses LSD as a psychedelic substance, salts thereof, tartrates thereof, analogs thereof, homologues thereof, or any ergotamine. Other psychedelics can be used in the methods of the present invention that are tryptamines or phenethylamines and induce the same or similar acute effects as LSD on the 5D-ASC scale such as, but not limited to, psilocybin, mescaline, dimethyltryptamine (DMT), 2,5-dimethoxy-4-iodoamphetamine (DOI), 2,5-dimethoxy-4-bromoamphetamie (DOB), salts thereof, tartrates thereof, analogs thereof, or homologues thereof.

LSD is preferably administered in a dose of 200 μg, but a range of 25-400 μg can also be used. Preferably, two doses of LSD are given. A second dose of typically 200 μg (25-400 μg) can be administered between 4 and 5 weeks after the first dose. Effects of the LSD can last 8-12 hours after administration, and the individual is preferably supervised by medical personnel such as a psychiatrist during this time. Other psychedelics can be dosed by one skilled in the art.

LSD is preferably administered orally but nasal, transdermal, subcoutaneous, intravenous, and intramuscular formulations may also be suitable.

A single dose can also be administered and provide effects. Additional non-drug sessions following an LSD session with the present invention and in patients with anxiety disorder may be beneficial if performed by the same therapist and within the same setting or if a placebo or lower dose of LSD is used due to the presence of effect conditioned by the first LSD administration.

Based on phase 1 and dose-finding studies described above, a higher 200 μg dose was selected to be used in the present invention in patients as similarly done in the pilot study in patients (Gasser et al., 2014; Gasser et al., 2015). However, it should be noted that during the dose-finding process and with more pharmacokinetic data becoming available (Holze et al., 2020a; Holze et al., 2021), it became clear that past studies used 140 μg of LSD rather than the reported 200 μg. In fact, another advantage of the present invention is that it used pharmaceutically defined doses of LSD for the first time in patients while the past studies used non-defined doses, meaning that the content and the content uniformity of LSD in the doses was not clear and later studies even documented an approximately 30% lower LSD content (Holze et al., 2019) than reported in the original publications (Gasser et al., 2014; Gasser et al., 2015). Importantly, also older studies in healthy subjects used doses of LSD that were not pharmaceutically defined (Dolder et al., 2015b; Dolder et al., 2016; Schmid et al., 2015) in contrast to the doses used in the present invention (Holze et al., 2019).

As described above, psychedelics including LSD, psilocybin and plant-derived ayahuasca containing DMT have been used in treating individuals with depression associated with life-threatening illnesses. However, there have been no studies using LSD in patients with generalized anxiety disorder or any other anxiety disorder that is not linked to a life-threatening illness and thus not an adjustment disorder-type of anxiety. In fact, patients with anxiety or affective disorders within 1 year prior to the onset of cancer were typically excluded in past studies evaluating effect of psychedelics in patients with cancer and related anxiety (Grob, 2011 #1910; Gasser, 2015 #3955).

Cancer and other life-threatening diseases may result in an adjustment disorder-type of anxiety. This type of anxiety was not present in the patient before the somatic illness and developed as a result of the real threat to life caused by the cancer or life-threatening illness. The psychedelic therapy aims for a reduction of the cancer-related anxiety and is designed to help with an existential crisis and often aimed at reducing of fear of dying (Grob, 2011 #1910; Gasser, 2015 #3955). In contrast, in forms of anxiety that have no physical cause for the fear such as generalized anxiety disorder, social anxiety disorder (social phobia), panic disorder, and/or agoraphobia there is no obvious external cause of the disorder. The patient is in distress for “no obvious reason” and there may be severely impaired functioning in the absence of an illness-causing reason. Such types of anxiety have in the past also been termed endogenous and coming from within the psyche in contrast to the exogenous type of anxiety caused by another cause external to the psyche.

In Example 1, in a study in human patients with generalized anxiety disorder, the present innovation was compared for the first time with placebo in a double-blind placebo-controlled, randomized trial including patients with generalized anxiety disorder in the absence of severe somatic illness. The study in the Example documented significant beneficial effects on mood and on psychological distress and trend improvements on ratings of anxiety. LSD significantly reduced state-trait anxiety inventory (STAI)-S, STAI-T, and STAI-G ratings after a second dose. STAI-S and STAI-G ratings were already significantly reduced after a first dose. LSD significantly reduced scores after the second dose on the HDRS, the BDI, and the SCL-90 Global. LSD induced significant and marked alterations in all scales of the 5D-ASC questionnaire. LSD significantly and strongly increased ratings of mystical-type experiences on the MEQ30 questionnaire. Good drug effects of LSD are predictive of good therapeutic outcomes two weeks after treatment.

The method can also reduce psychological distress and/or increase quality of life in the individual. The method can also enhance psychotherapy that the individual receives (such as administered on separate days before and after the psychedelic administration). The method can be used when the individual has an insufficient therapeutic response or adverse effects after the use of other psychedelics substances and the method can be used as a second-line treatment. The method can also be used when the individual has a need for a qualitatively different psychedelic response after the use of other psychedelics substances.

The present invention uses LSD to induce a psychedelic state and to assist psychotherapy and provides the first data supporting the use of LSD in generalized anxiety disorder to improve mood, symptoms of psychological distress, and/or quality of life and to provide a medical benefit to these patients and society.

The compounds of the present invention are administered and dosed in accordance with good medical practice, taking into account the clinical condition of the individual patient, the site and method of administration, scheduling of administration, patient age, sex, body weight and other factors known to medical practitioners. The pharmaceutically “effective amount” for purposes herein is thus determined by such considerations as are known in the art. The amount must be effective to achieve improvement including but not limited to improved survival rate or more rapid recovery, or improvement or elimination of symptoms and other indicators as are selected as appropriate measures by those skilled in the art.

In the method of the present invention, the compounds of the present invention can be administered in various ways. It should be noted that they can be administered as the compound and can be administered alone or as an active ingredient in combination with pharmaceutically acceptable carriers, diluents, adjuvants and vehicles. The compounds can be administered orally, subcutaneously or parenterally including intravenous, intramuscular, and intranasal administration. The patient being treated is a warm-blooded animal and, in particular, mammals including humans. The pharmaceutically acceptable carriers, diluents, adjuvants and vehicles as well as implant carriers generally refer to inert, non-toxic solid or liquid fillers, diluents or encapsulating material not reacting with the active ingredients of the invention.

The doses can be single doses or multiple doses or a continuous dose over a period of several hours.

When administering the compound of the present invention parenterally, it will generally be formulated in a unit dosage injectable form (solution, suspension, emulsion). The pharmaceutical formulations suitable for injection include sterile aqueous solutions or dispersions and sterile powders for reconstitution into sterile injectable solutions or dispersions. The carrier can be a solvent or dispersing medium containing, for example, water, ethanol, polyol (for example, glycerol, propylene glycol, liquid polyethylene glycol, and the like), suitable mixtures thereof, and vegetable oils.

Proper fluidity can be maintained, for example, by the use of a coating such as lecithin, by the maintenance of the required particle size in the case of dispersion and by the use of surfactants. Nonaqueous vehicles such a cottonseed oil, sesame oil, olive oil, soybean oil, corn oil, sunflower oil, or peanut oil and esters, such as isopropyl myristate, may also be used as solvent systems for compound compositions. Additionally, various additives which enhance the stability, sterility, and isotonicity of the compositions, including antimicrobial preservatives, antioxidants, chelating agents, and buffers, can be added. Prevention of the action of microorganisms can be ensured by various antibacterial and antifungal agents, for example, parabens, chlorobutanol, phenol, sorbic acid, and the like. In many cases, it will be desirable to include isotonic agents, for example, sugars, sodium chloride, and the like. Prolonged absorption of the injectable pharmaceutical form can be brought about by the use of agents delaying absorption, for example, aluminum monostearate and gelatin. According to the present invention, however, any vehicle, diluent, or additive used would have to be compatible with the compounds.

Sterile injectable solutions can be prepared by incorporating the compounds utilized in practicing the present invention in the required amount of the appropriate solvent with various of the other ingredients, as desired.

A pharmacological formulation of the present invention can be administered to the patient in an injectable formulation containing any compatible carrier, such as various vehicle, adjuvants, additives, and diluents; or the compounds utilized in the present invention can be administered parenterally to the patient in the form of slow-release subcutaneous implants or targeted delivery systems such as monoclonal antibodies, vectored delivery, iontophoretic, polymer matrices, liposomes, and microspheres. Examples of delivery systems useful in the present invention include: U.S. Pat. Nos. 5,225,182; 5,169,383; 5,167,616; 4,959,217: 4,925,678; 4,487,603; 4,486,194; 4,447,233; 4,447,224; 4,439,196; and 4,475,196. Many other such implants, delivery systems, and modules are well known to those skilled in the art.

The present invention also provides for a method of treating anxiety, by administering a psychedelic to an individual having anxiety not associated with causes such as a life-threatening serious somatic illness, and inducing positive acute drug effects and positive long-term therapeutic effects in the individual. As shown in Example 1, LSD induced significant and marked alterations in all scales of the 5D-ASC questionnaire and LSD significantly and strongly increased ratings of mystical-type experiences on the MEQ30 questionnaire (showing acute effects). These acute effects of LSD on the 5D-ASC and MEQ30 questionnaires were associated with the therapeutic effects of LSD 2 weeks after the second administration (showing long-term therapeutic effects).

The invention is further described in detail by reference of the following experimental study example. The example is provided for the purpose of illustration only and is not intended to be limiting unless specified. Thus, the invention should in no way be construed as being limited to the example, but rather, should be construed to encompass any and all variations which become evident as a result of the teaching provided herein.

### Example 1

A randomized, double-blind, placebo-controlled phase II study was performed with LSD treatment in psychiatric anxiety disorder patients or in persons suffering from anxiety symptoms in association with a severe somatic disease.

The study included both patients with and without somatic disease. The data from the patients without somatic disease is shown to illustrate the use of the present invention in generalized anxiety disorder and thus in a form of anxiety not linked to cancer or fear of dying.

Specifically, in patients with generalized anxiety disorder, LSD or placebo was administered in double-blind manner in two sessions separated by 6 weeks and anxiety, depression and psychological distress were assessed between sessions, 2, 8, and 16 weeks after the second session. Then patients who received LSD crossed over to placebo and vice versa.

Thus, the study allowed evaluating the effect of LSD against placebo between-subjects in the first period (parallel design) and within-subjects against placebo over the entire study duration (cross-over design).

LSD had beneficial effects on anxiety, depression, and quality of life in these patients with generalized anxiety disorder as described in detail below.

### Study Introduction

Lysergic acid diethylamide (LSD) is a prototypical classic hallucinogen (Nichols, 2004; Passie et al., 2008). LSD was first synthesized by the Sandoz chemist Albert Hofmann who also discovered its psychotropic effects (Hofmann, 1979). In the 1950s to 1970s, LSD was initially used as an experimental tool (“psychotomimetic”) to study psychotic-like states and model psychosis (Bercel et al., 1956; Koelle, 1958) and as an adjunct in “psycholytic (substance-assisted) psychotherapy.”

LSD has been investigated for the treatment of alcoholism (Krebs et al., 2012), addiction (Savage et al., 1973), cluster headache (Sewell et al., 2006), and anxiety associated with terminal illness (Gasser et al., 2014; Grof et al., 1973; Pahnke et al., 1969). LSD became a well-studied substance with several thousands of early scientific reports (Hintzen et al., 2010; Nichols, 2004; Nichols, 2016; Passie et al., 2008).

Today, LSD is illicitly-used for recreational (personal or spiritual) purposes. It is estimated that 38 million US people or 15% over the age of 12 ingested a hallucinogen once in their lifetime (Johnston et al., 2016; Krebs et al., 2013a). In Europe, the lifetime prevalence of LSD use among adults is estimated to be in the range of 6-8%. Thus, a significant proportion of the western society is familiar with the effects of this substance.

LSD is not associated with compulsive drug seeking (addiction) and there are relatively few medical emergencies and adverse effect (Nichols, 2016). Use of LSD or psilocybin is not associated with mental health problems and may even be protective (Johansen et al., 2015; Krebs et al., 2013b). Despite LSD's widespread recreational use clinical research using LSD came to a halt in the 1970ies due to political and cultural pressures and regulatory restrictions.

Recently, the medical value of hallucinogens has again been studied in several clinical trials (Baumeister et al., 2014: Bogenschutz et al., 2015; Davenport, 2016; Gasser et al., 2014; Gasser et al., 2015; Grob et al., 2011; Johnson et al., 2014; Kupferschmidt, 2014; Nichols, 2016). Specifically, LSD and psilocybin were both shown to reduce anxiety associated with life-threatening diseases (Gasser et al., 2014; Gasser et al., 2015; Grob et al., 2011). Based on these preliminary data, small numbers of patients are currently treated with LSD by specialized psychiatrists in Switzerland in the context of compassionate use with special case-by-case authorizations by the Federal Office of Public Health (BAG).

The currently available pilot study data are insufficient in amount and quality and need to be confirmed in larger and placebo-controlled studies. Therefore, the present study example aims at evaluating the effects of LSD compared with placebo on anxiety and depression in patients with anxiety disorder (novel use) or/and increased anxiety associated with life-threatening illness (past use, confirmatory data).

The study was conducted in collaboration with the psychiatrist Dr. P. Gasser, who conducted a pilot phase II type study on the safety and efficacy of LSD-assisted psychotherapy in patients (Gasser et al., 2014; Gasser et al., 2015).

The study was financially supported by the Swiss Medical Society for Psycholytic Therapy (Schweizerische Ärztegesellschaft für Psycholytische Therapie, SAEPT) and the University Hospital Basel.

### Objectives of the Example Study

The goal of the present innovation was to explore 1) reduction in anxiety (STAI), 2) reduction in depression (HDRS and BDI), and 3) improvements in general psychopathological symptoms (SCL-90), and to document safety.

### Study Methods

### Study Design

The examples study used a double-blind cross-over design with two treatment sequences each lasting 24 weeks (LSD vs. placebo-assisted psychotherapy) and separated by a 2-week between-sequence period. Order was counter-balanced and random. Each participant served as its own control over the 52-weeks total study duration. Treatment effects are also compared between subjects for the first 24 week study period before the cross-over.

### Study Duration

The study ran from Jan. 1, 2017 to Dec. 31, 2021 (to Dec. 31, 2020, for patients with generalized anxiety disorder); duration per participant: 52 weeks including screening and with a follow-up at 102 weeks.

### Study Sites

1) Ambulatory study center, University Hospital of Basel, 2) Private psychiatry practice Dr. Gasser, Solothurn.

### Study Investigators/Personnel

The LSD-/placebo sessions and the study visits are conducted by study physicians/psychiatrists. In general one physician conducts the sessions or visits and the same person treats the same patient throughout the entire study.

### Study Population

Anxiety with Vs. Without Life-Threatening Disease

The goal was to include patients with anxiety disorders with or without life-threatening diseases. Study patients needed to meet diagnostic criteria for DSM anxiety disorder or report a score greater than 40 on the state or trait STAI scale (Spielberger et al., 1983). Patients without life-threatening diseases always needed to meet a DSM-V diagnosis of anxiety disorder (elevated STAI anxiety alone was not sufficient for study inclusion in these patients). The aim was to include approximately equally large subgroups of patients with anxiety with and without life-threatening diseases.

### Recruitment

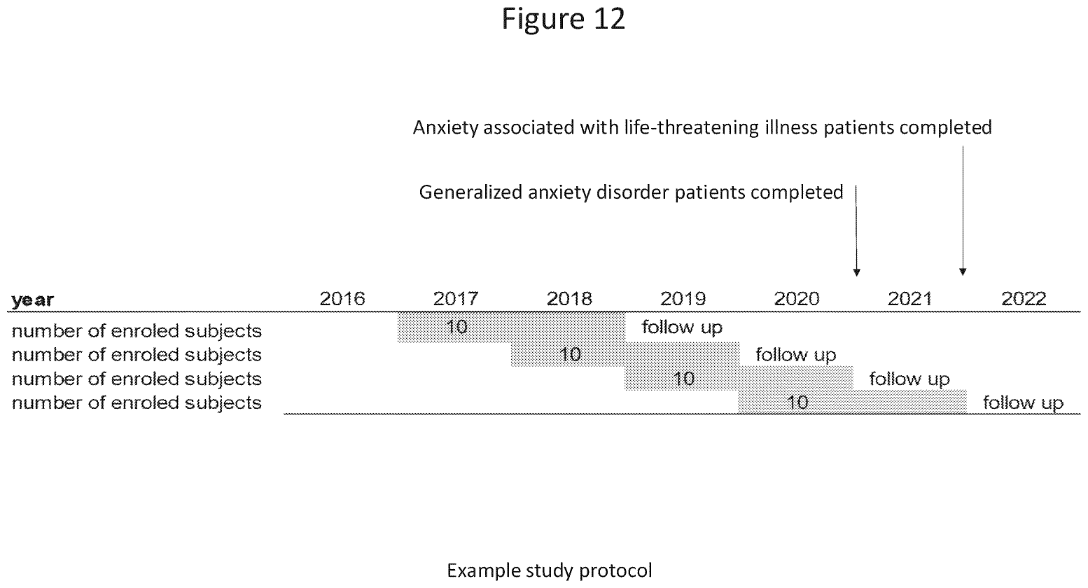

Patients were recruited mostly form patients of the participating study psychiatrists (private practice Dr. Gasser). 40 participants were enrolled in the study. Drop outs during the study were replaced to reach a final study sample of at least 30 subjects who completed the study (12 months). Approximately ten participants were recruited per year resulting in a recruitment period of 4 years and a total duration of 5 years (FIG. 12).

### Inclusion Criteria

1. Age>25 years.
2. Meet DSM-IV criteria for anxiety disorder as indicated by the SCID-IV or have a score of at least 40 on the state- or trait STAI scale at study inclusion.
3. 40% or more of the participants should have a diagnosis of advanced-stage potentially fatal illness (autoimmune, neurological, or cancer without CNS involvement). Patients should be ambulatory and not terminal and likely to have a roughly estimated life expectancy of greater than twelve months.
4. Patients without advanced-stage potentially fatal illness need to meet DSM-IV criteria for anxiety disorder (elevated STAI score not sufficient for inclusion).
5. Sufficient understanding of the study procedures and risks associated with the study.
6. Participants must be willing to adhere to the study procedures and sign the consent form.
7. Participants are willing to refrain from taking any psychiatric medications during the experimental session period. If they are being treated with antidepressants or are taking anxiolytic medications on a fixed daily regimen such drugs must be discontinued long enough before the LSD/placebo treatment session to avoid the possibility of a drug-drug interaction (the interval will be at least 5 times the particular drug's half-life [typically 3-7 days]).
8. If in ongoing psychotherapy, those recruited into the study may continue to see their outside therapist, provided they sign a release for the investigators to communicate directly with their therapist. Participants should not change therapists, increase, or decrease the frequency of therapy or commence any new type of therapy during the study (not including the follow-up).
9. Participants must also refrain from the use of any psychoactive drugs, with the exception of the long term pain medication or caffeine or nicotine, within 24 hours of each LSD/placebo treatment session. They must agree not to use nicotine for at least 2 hours before and 6 hours after each dose of LSD. They must agree to not ingest alcohol-containing beverages for at least 1 day before each LSD treatment session. Non-routine medications for treating breakthrough pain taken in the 24 hours before the LSD treatment session may result in rescheduling the treatment session to another date, with the decision at the discretion of the investigators after discussion with the participant.
10. Participants must be willing not to drive a traffic vehicle or to operate machines within 24 hours after LSD/placebo administration.

### Exclusion Criteria

1. Women who are pregnant or nursing, or of child bearing potential and are not practicing an effective means of birth control (double-barrier method, i.e. pill/intrauterine device and preservative/diaphragm).
2. Past or present diagnosis of a primary psychotic disorder. Subjects with a first degree relative with psychotic disorders are also excluded.
3. Past or present bipolar disorder (DSM-IV)
4. Current substance use disorder (within the last 2 months, DSM-V, except nicotine).
5. Somatic disorders including CNS involvement of the cancer, severe cardiovascular disease, untreated hypertension, severe liver disease (liver enzymes increase by more than 5 times the upper limit or normal) or severely impaired renal function (estimated creatinine clearance <30 ml/min), or other that in the judgement of the investigators pose too great potential for side effects.
6. Weight less than 45 kg.
7. Suicide risk or likely to require psychiatric hospitalization during the course of the study
8. Requiring ongoing concomitant therapy with a psychotropic drug (other than as needed, anxiety medications, and pain control medications) and unable or unwilling to comply with the washout period.

### Schedule of Events

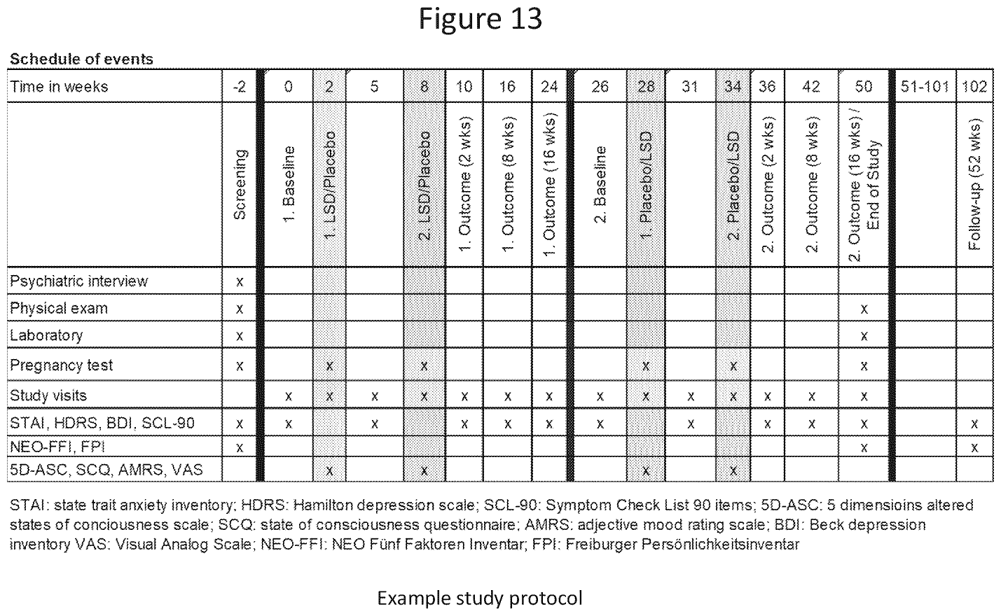

The schedule of events for a participant is shown in FIG. 13. Over the course of 52 weeks, participants took part in a 2 hour screening visit, four 11-13 hour LSD/placebo treatment sessions, 10 1 hour study visits, and a 1 hour end of study visit. A follow-up (not part of the clinical study duration) will also be performed using questionnaires sent by mail.

### Screening Procedure

### Informed Consent

The subjects were informed about the study procedures and associated risks both verbally and by the approved written consent form. The study physician and the subject both personally signed and dated the consent form as confirmation of consent.

### Physical Health

Subjects were examined by a study physician including medical history, physical examination, vital signs, and blood chemistry. Body weight and height were also be measured.

### Mental Health

Subjects were screened using a SCID for DSM-IV (Wittchen et al., 1997). The psychiatric interview was conducted by a study psychiatrist who also decided whether subjects met the psychiatric inclusion criteria. The psychiatric interview started with the SCID to provide a DSM-IV diagnosis of anxiety disorder and to rule out exclusionary Axis I diagnoses (i.e., current substance use disorder, psychotic disorder, bipolar disorder). The HDRS, BDI, STAI, and SCL-90 were then completed.

### History of Substance Use and Urine Drug Screens

The history of substance was recorded during the screening visit. As in a previous similar study (Gasser et al., 2014), it was expected that most participants would not have experience with hallucinogenic drugs. Similarly, psilocybin has typically been used in subjects with little to no previous experience (Griffiths et al., 2008; Griffiths et al., 2011; Griffiths et al., 2006; Johnson et al., 2014; MacLean et al., 2011). Substance use disorder in the past (not within the last 2 months) was not an exclusion criterion if no severe adverse reactions occurred to hallucinogens. Subjects were asked to abstain from any illicit drug use during the study and drug screens were performed before test-sessions. Positive urine drug screens (with the exception of tetrahydrocannabinol (Northcote) or opioids if used for pain management) resulted in postponing of the study session. This is because THC consumption can be detected in urine for up to several weeks and use days before a study day is unlikely to affect the outcome. Based on the pilot study data (Gasser et al., 2014) we expected no positive urine drug screens. Subjects were also be asked to abstain from excessive alcohol consumption (not more than 10 drinks/week) and to abstain on the day before the test sessions.

### Screening Laboratory Tests

A routine laboratory blood test was performed at the screening examination including creatinine, and ALAT.

### Personality

Personality traits are known to affect subjective responses to psychoactive substances (Studerus et al., 2012; White et al., 2006). The NEO-five factor inventory (NEO-FFI) and the Freiburger Personality Inventory (FPI) were administered during the screening visit to assess personality traits and their potential modulatory effects on the response to LSD in this study. The same instruments are being used in experimental studies in healthy subjects. We will explore whether personality traits alter the effects of LSD or are altered by LSD (Carhart-Harris et al., 2016b; Griffiths et al., 2008; MacLean et al., 2011).

### LSD/Placebo Sessions

Each of the four LSD/Placebo sessions lasted 12 hours from 8 AM to 8 PM. Sessions took place in a quiet room at the Ambulatory Study Center of the University Hospital Basel (Basel center) or in the private practice office of Dr. P. Gasser (Solothum center). At the beginning of the session, current mood and mental state were discussed and a urine drug screen was taken and any AEs since the last visit were recorded. Any open questions or concerns were addressed by the investigator. The participants were advised to lie in a bed or sit comfortably on a chair. Other than going to the bathroom, the participants remained in the treatment room for the entire 12-hour experimental session and were supervised constantly up to 8-12 hours after LSD/placebo administration depending on the participant's response and as needed. The subjective effects of LSD at the dose used in this study were expected to last 8-12 hours (Gasser et al., 2014; Holze et al., 2021; Schmid et al., 2015). The dose was relatively high and has been shown to produce the full spectrum of a typical LSD experience, without full dissolution of normal ego structures (Gasser et al., 2014; Gasser et al., 2015; Holze et al., 2021; Schmid et al., 2015). During the LSD/placebo effect, the participants were instructed to focus their awareness inward. Subjects were encouraged to wear eye shades during the first hours (or the light was dimmed) as well as to listen to music and discussions/talking much with the investigator were discouraged. Subjects were allowed to remain mostly undisturbed with brief physical contacts every two hours when heart rate and blood pressure were measured. At the end of the sessions, 10-12 hours after LSD/placebo administration, the acute subjective peak effects were retrospectively rated by the participants using the 5D-ASC, SCO, AMRS, and VASs and the experience was discussed. Ten-twelve hours after LSD/placebo administration the participants were allowed to return to their home with company and supervision provided by the partner, a relative, or a friend. If supervision was not available or if effects persisted, the night was spent at the study site.

### Study Visits

Preparatory study visits of 60-90 minutes served to discuss the participant's history (life narrative), personality, health, present social and emotional situation (meaning-centered) and concerns as well as to explain the action of LSD, answer questions, and prepare for the LSD-/placebo-assisted sessions (intentions, expectations (Johnson et al., 2008). Additional important goals of the meetings included establishing a comfortable level of rapport and trust between the patient and the investigators. The patient's anxiety and personal situation were reviewed (somatic disease-related or non-somatic) and it was discussed what might occur during the experimental session. One preparatory visit was scheduled 2 weeks prior to the first LSD/Placebo session and one visit between the two LSD/Placebo sessions, which were separated by 6 weeks. Visits after the substance-assisted sessions served to discuss and integrate the experiences of the patient during the session. There was no formal guideline for these sessions (making meaning together, elements of psycholytic therapy, reuse of music played during the session; (Breitbart et al., 2014; Johnson et al., 2008; Leuner, 1969). The patients were also motivated to write about their experience and their reports were then also discussed during the sessions. Three meetings were scheduled 2, 8, and 16 weeks after the two LSD/Placebo sessions and included the assessments of the outcomes. This schedule of 10 study visits represented the minimum number of non-substance meetings. Additional meetings were scheduled as needed but were not mandatory and were only performed if therapeutically required. The number of meetings was to be similar in both treatment periods. Thus, if additional meetings were held in the first treatment period of the study, additional meetings were also scheduled in the second treatment period at the corresponding times. If additional meetings were held only in the second study period the number of meetings and reasons have to be noted on the case report form (CRF). The times (weeks) at which the sessions were to take place according to the treatment schedule was to be adhered to as closely as possible (+/−1-3 weeks) but deviation was not considered protocol violation. Actual dates of all meetings were recorded on the CRF. The number and general content of the sessions was largely standardized in the present study and the same study physician cared for a patient throughout the entire study. Differences between the investigators and the patient-investigator interaction were reduced by the cross-over design of the study also comparing the effects of LSD and placebo within-subjects.

### Concomitant Medication Use and Other Therapy

Many interactions of chronic medications with acute administration of LSD have been studied (Hintzen et al., 2010; Passie et al., 2008). Concomitant medications were recorded at the screening visit and before each visit/study session. Routine medication (drugs for hypertension, aspirin, statins, analgesics) were in general continued during the study while antidepressants had to be paused before the LSD/placebo sessions. The literature and clinical pharmacological judgement was used to decide if a chronic medication needed to be paused additionally to the situations noted in the protocol: Antidepressants: Selective serotonin reuptake inhibitors (SSRIs) may attenuate the response to LSD (Bonson et al., 1996a; Bonson et al., 1996b; Strassman, 1992). Lithium or tricyclic antidepressants may enhance the effects of LSD (Bonson et al., 1996b). Participants using any of these drugs as chronic medications were required to taper off antidepressant and anti-anxiety medications at least five half-lives before each LSD/placebo session. Typically, this resulted in a one-week pause. This approach was used before in two of 12 patients in the pilot study (Gasser et al., 2014). This approach was used in clinical studies with psilocybin where drugs for depression and anxiety were stopped two weeks before the administration of psilocybin and were even not reinstalled after because of the marked therapeutic responses (Carhart-Harris et al., 2016b; Griffiths, 2016). Subjects who required ongoing treatment with antidepressants were excluded from the present study. Anxiolytics: Any anxiolytic treatment with benzodiazepines were continued during the study as needed. On the study days and during the LSD/placebo sessions the use of anxiolytics was discouraged but was allowed in the case of anxiety that could not be treated with verbal support. Analgesics: Any chronic pain medications was continued during the study including the LSD/placebo sessions as needed. Other ongoing psychotherapy could be continued but the number of sessions was not be increased or decreased and no new psychotherapy was to be started during the study period.

### Assessments and Measures

### Psychometric Assessments

### State-Trait Anxiety Inventory (STAI)

The STAI is a widely used self-report instrument for assessing anxiety in adults. It includes separate measures of state and trait anxiety (Spielberger et al., 1983). The STAI evaluates the essential qualities of feelings of apprehension, tension, nervousness, and worry. The STAI differentiates between the temporary condition of state anxiety and the more general and long-standing quality of trait anxiety. The STAI state anxiety subscale asks for feelings at the moment of filling out the questionnaire, and the STAI trait anxiety subscale ask subjects to indicate how they generally view themselves. For both subscales, scores from 20 to 39 represent mild anxiety, and scores from 40 to 59 indicated moderate anxiety, whereas scores from 60 to 80 indicated severe anxiety. Both the state and trait STAI are commonly used as outcome measures in studies in patients with anxiety disorder (Fisher et al., 1999; Laakmann et al., 1998). A global STAI score can be derived by adding up the state and trait anxiety scale scores (range: 40-160 points). Similar to the pilot study both STAI scale scores were used to determine study inclusion at the screening visit (Gasser et al., 2014; Gasser et al., 2015). Also similar to the pilot study and a similar trial using psilocybin (Grob et al., 2011) the STAI was the main outcome measure for this study. The pilot study showed within-subjects reductions in STAI state and trait anxiety at 2 months after two LSD-assisted psychotherapy sessions in patients with life-threatening diseases (Gasser et al., 2014; Gasser et al., 2015) with comparable responses in both scales. However, no adequately sized placebo group was included. Reductions in the STAI trait scale over time at 4 and 12 weeks were seen in a pilot study of psilocybin treatment for anxiety with advanced-stage cancer (Grob et al., 2011). Psilocybin non-significantly reduced state and trait STAI scores compared with placebo at 2 weeks after verum compared with placebo administration (Grob et al., 2011). However, the study included only 12 subjects and a placebo condition only up to 2 weeks. In the present study, both STAI scales were administered at screening, 2 weeks before. 2, 8, and 16 weeks after LSD and placebo administration and at follow-up. The scoring was performed according to (Spielberger et al., 1983) and performed at screening and also implemented in the clinical data base used (SecuTrial) for this study.

### Hamilton Depression Rating Scale (HDRS)

The study psychiatrists assessed the patient's depression severity with the HDRS (Hamilton, 1960: Hamilton, 1980). This rating scale consists of 21 items (3- to 5-point ratings) asking about symptoms related to depression such as low mood, suicidality, irritability, tension, loss of appetite, insomnia, loss of interests, somatic symptoms, and similar. The summary scores were calculated as described (Hamilton, 1960) and implemented on the clinical data base (SecuTrial).

### Beck Depression Inventory (BDI)

The BDI consists of 21 questions developed to measure the severity of depression (Beck et al., 1961). The German BDI-II version (Hautzinger et al., 2006; Kuhner et al., 2007) was used as a self-assessment. The BDI previously revealed an improvement of mood 6 months after psilocybin-assisted psychotherapy for anxiety in patients with advanced-stage cancer (Grob et al., 2011). The summary scores were calculated as described (Hautzinger et al., 2006) and implemented on the clinical data base (SecuTrial).

### Symptom-Check-List-90-R (SCL-90-R)

The SCL-90-R is a widely used psychological status symptom inventory (Derogatis et al., 1976; Schmitz et al., 2000) to assess overall psychological distress. We used the German version (Schmitz et al., 2000). Outcome measures were the global severity index, the positive symptom distress index, and the positive symptom total. Reductions in these SCL-90 scores were observed after LSD-assisted psychotherapy in patients with life-threatening illness (Gasser et al., 2014). SCL-90 scores were calculated according to (Franke, 2002).

### Altered States of Consciousness (5D-ASC)

The 5 dimensions of altered states of consciousness (5D-ASC) scale is a visual analog scale consisting of 94 items (Dittrich. 1998; Studerus et al., 2010). The instrument is constructed of five scales and allows assessing mood, anxiety, derealization, depersonalization, changes in perception, auditory alterations, and reduced vigilance. The scale is well-validated (Studerus et al., 2010) and has been used to characterize the acute subjective effects of LSD in experimental studies in healthy subjects (Schmid et al., 2015) and in patients (Gasser et al., 2014; Gasser et al., 2015). The 5D-ASC scale was administered once at the end of each session and subjects are instructed to retrospectively rate peak alterations during the study session. Each item of the scale was scored on a 0-100 mm VAS. The attribution of the individual items to the subscales of the 5-ASC was according to (Dittrich, 1998; Studerus et al., 2010) and the new subscales were defined as published (Dittrich, 1998; Studerus et al., 2010). The link of the items to the subscales was implemented in the clinical data base (SecuTrial). An association of the acute peak response to psilocybin and the long-term therapeutic effects of psilocybin have repeatedly been documented (Carhart-Harris et al., 2016a; Griffiths, 2016). It is hypothesized that mystical-type experiences critically contribute to the therapeutic potential of LSD and psilocybin (Griffiths, 2016). Aspects of this (mystic) peak experience include an experience of unity (internal, external, perceiver-perceived), transcendence of time/space (eternity/infinity), beauty, sacredness/awsomness, deeply-felt positive mood, ineffiability (impossible to explain in words), and paradoxicality (died but never felt so alive at the same time) (Barrett et al., 2015; Griffiths et al., 2008; MacLean et al., 2011; MacLean et al., 2012). On the other hand LSD alters emotion processing in ways that may also contribute to its potential therapeutic effects (Dolder et al., 2016).

### States of Consciousness Questionnaire (SCQ)

This 100-item questionnaire is rated on a six-point scale (Griffiths et al., 2011; Griffiths et al., 2006) and has been used with psilocybin (Griffiths et al., 2006) and LSD (Gasser et al., 2014). The scale has been used to assess mystical experiences in studies using psilocybin (Griffiths et al., 2011: Griffiths et al., 2006) and to explore associations between such experiences and positive long-term effects of psilocybin. The SCQ was administered once at the end of each session and subjects are instructed to retrospectively rate peak alterations during the study session. As in previous studies with psilocybin, criteria for a “complete” mystical experience were scores on each of the following six scales of at least 60%: external or internal unity, sense of sacredness, noetic quality, transcendence of time, positive mood, and ineffiability. Data on each domain scale were expressed as a percentage of the maximum possible score. The link of the items to the subscales was implemented in the clinical database (SecuTrial).

### Adjective Mood Rating Scale (AMRS)

The AMRS or EWL60S is a 60-item Likert scale that allows repeated assessment of mood in 6 dimensions: activation, inactivation, well-being, anxiety/depressed mood, extro- and introversion, and emotional excitability. The German EWL60S version is used (Janke et al., 1978). The AMRS was administered once at the end of each session and subjects were instructed to retrospectively rate peak alterations during the study session. The scoring into the subscales was performed according to (Janke et al., 1978) and was implemented in the clinical data base (SecuTrial).

### Visual Analog Scales (VASs)

At the end of the sessions, a set of VASs will be used to rate the effects of LSD/placebo over the entire session (with reference to the peak effects). VASs included ratings of “any drug effect”, “good drug effect”. “bad drug effect”, “anxiety”, “happy”, and “open” as previously used (Schmid et al., 2015).

### Adverse Effects

Subjects were asked to report any adverse events (AEs) during the sessions or between study sessions at the beginning of the next session/visit and at the end of study (EOS) visit. The investigator rated hallucinogen-specific AEs that occurred during the session using a checklist adapted from (Gasser et al., 2014; Griffiths et al., 2006; Schmid et al., 2015). Peak effects were rated at the end of the session based on the patients description and investigators' observation by the investigator as “not reported”, “mild”, “moderate”, or “severe” for: headache, dizziness, dry mouth, nausea, anxiety, paranoid thinking, emotional distress, emotional lability, blurred vision, chills/feeling cold, impaired balance. Heart rate and blood pressure were measured at 2-hourly intervals (Grob et al., 2011).

### End of Study (EOS) Visit

At the end of the study the study physician repeated the physical examination and blood chemistry. Adverse events were recorded.

### Long-Term Follow-Up

A follow-up (not part of the clinical study) is conducted by mail 52 weeks after study completion as similarly performed in the pilot study (Gasser et al., 2015). Participants are asked to indicate any beneficial or adverse lasting effects. The STAI, BDI, and SCL-90 are repeated. Additionally, the personality questionnaires used during the screening (NEO-FFI and FPI are repeated to evaluate potential changes in personality (Carhart-Harris et al., 2016b; MacLean et al., 2011). Additionally, the 143-item Johns Hopkins University Persisting Effects Questionnaire is used which seeks information about changes in attitude, moods, behavior, and spiritual experience (Griffiths et al., 2011; Griffiths et al., 2006). 140 items are rated on a six-point scale and include attitudes about life (13 positive and 13 negative items), attitudes about self (11 positive and 11 negative items), mood changes (nine positive and nine negative items), behavioral changes (one positive and one negative item), spirituality (22 positive and 21 negative items). Three additional questions are included: 1. How personally meaningful was the experience?2. Indicate the degree to which the experience was spiritually significant for you?3. Do you believe that the experience and your contemplation of the experience have led to change in your current sense of personal well-being or life satisfaction?

### Study Drug

Recreational use of LSD: LSD is used for recreational (personal or spiritual) purposes. It is estimated that 38 million US people or 15% over the age of 12 ingested a hallucinogen once in their lifetime (Krebs et al., 2013a). LSD is the most widely used hallucinogenic drug; 24 million Americans used LSD at least once in their lifetime (Johnston et al., 2016; Krebs et al., 2013a). Thus, a significant proportion of the western society is familiar with the effects of this substance.

Pharmacology of LSD: LSD is a partial 5-HT_{2A} receptor agonist LSD (Nichols, 2016; Rickli et al., 2015; Rickli et al., 2016). LSD also stimulates 5-HT₁ receptors, adrenergic α₁ receptors and dopaminergic D₁₋₃ receptors (Rickli et al., 2015). However, these receptor interactions are considered less relevant for the psychotropic action of LSD (Nichols, 2016). The subjective effects of hallucinogens are generally considered to be mediated primarily by activation of the 5-HT_{2A} receptor (Nichols, 2016; Vollenweider et al., 1998).

Dose selection for this study: The present study uses a dose of 200 μg of LSD hydrate (ethanolic solution, per os) similar to the pilot study (Gasser et al., 2014; Gasser et al., 2015). This dose corresponds to a moderate-high dose in humans (Passie et al., 2008). The same dose has also been used in the laboratory in healthy subjects (Dolder et al., 2015a; Dolder et al., 2015b; Holze et al., 2021; Schmid et al., 2015).

Clinical pharmacology of LSD: The LSD effects (200 μg) peak at 2 hours and last up to 12 hours after administration (Dolder et al., 2015b; Holze et al., 2021; Schmid et al., 2015). The plasma elimination half-life of LSD is 3 to 3.6 hours (Aghajanian et al., 1964; Dolder et al., 2015b; Holze et al., 2019; Holze et al., 2020a; Holze et al., 2021).

Adverse effects of LSD in controlled studies: Perceptual changes after administration of LSD include illusions, pseudohallucinations, intensification of color perception, metamorphosis-like changes in objects and faces, kaleidoscopic or scenic visual imagery, synesthesia and alterations in thinking and time experience (Holze et al., 2021; Passie et al., 2008; Schmid et al., 2015). Body perception is altered involving changes in body image, unusual inner perception of bodily processes and metamorphic alterations of body contours (Holze et al., 2021; Passie et al., 2008; Schmid et al., 2015). At the dose of LSD to be used in the present study, subjects are expected to retain their thought control and in contrast to psychotic patients, subjects will remain aware of the transient state of the drug-induced experience. No complete loss of thought or body control was observed in studies using 200 μg LSD (Holze et al., 2021; Schmid et al., 2015). The subjective effects of LSD are experienced as overall positive in a controlled clinical setting and overall similar in healthy subjects and patients (Gasser et al., 2014; Gasser et al., 2015; Holze et al., 2021; Passie et al., 2008; Schmid et al., 2015; Schmid et al., 2021) but may include transient dysphoria, anxiety or mood swings. In the pilot study in patients, neither LSD nor placebo produced any drug-related severe adverse events, that is, no panic reactions, no suicidal crisis or psychotic state, and no medical or psychiatric emergencies requiring hospitalization. AEs included moderate anxiety (in 23% of the LSD sessions and in 50% of the placebo sessions), mild-to-moderate emotional distress (in 36% of the LSD sessions and in 33% of the placebo sessions, mild affect lability (in 14% of the LSD sessions and in 0% of the placebo sessions), moderately feeling cold (in 45% of the LSD sessions and in 0% of the placebo sessions), and mild gait disturbance (in 32% of the LSD sessions and in 0% of the placebo sessions). In a few instances some emotional distress persisted until the next day (Gasser et al., 2014). Mild irritation (not interfering with everyday performance) for a day or two after the LSD session was reported by some subjects (Gasser et al., 2015). No flashback phenomena were observed. In the 12-month follow up (Gasser et al., 2015), none of the participants reported lasting negative effects from the LSD sessions. Beyond the temporary difficulty reported by some in dealing with the initial effects of LSD (e.g. intense emotions, alteration in self-control), no AEs were mentioned consistent with previous findings (Cohen, 1960; Gasser, 1996). Acute AEs of LSD (200 μg) in healthy subjects in a controlled research setting included difficulty concentrating (number of subjects of 16 participants: 10 after LSD and 1 after placebo), headache (9 after LSD and 3 after placebo), dizziness (7 after LSD and 0 after placebo), nausea (4 after LSD and 1 after placebo), transient moderate anxiety (4 after LSD and 0 after placebo) (Schmid et al., 2015). Other researcher similarly report initial nausea, decreases in appetite, mild headache, dizziness, and trembling (Holze et al., 2021; Passie et al., 2008). LSD produced mild sympathomimetic stimulation including pupillary dilation (Passie et al., 2008; Schmid et al., 2015). There were no SAEs. The safety profile was confirmed in other studies at the Basel site in more then 50 healthy subjects (Dolder et al., 2016; Holze et al., 2021; Holze et al., 2020b). In laboratory studies using hallucinogens, mild or moderate anticipatory anxiety is common at the beginning of the onset of the drug effect (Griffiths et al., 2006). Dysphoria, anxiety and mild, transient ideas of reference/paranoid thinking may also occur in some subjects and can readily be managed with reassurance (Griffiths et al., 2006; Schmid et al., 2015). Negative experiences (bad trips) and flashback phenomena may occur in uncontrolled conditions (Strassman, 1984). On the other hand, under controlled and supportive conditions, the LSD experience reportedly had lasting positive effects (Carhart-Harris et al., 2016b). Similarly, psilocybin had persisting positive effects on attitudes, mood, and behavior (Griffiths et al., 2011; MacLean et al., 2011; Studerus et al., 2011). There are no lasting impairments in neurocognitive performance (Halpern et al., 1999). Epidemiological studies showed that psychiatric disorders are not increased in hallucinogen users (Johansen et al., 2015; Krebs et al., 2013b). LSD produces no neurotoxic effects also at very high doses.

Substance preparation and quality control: Analytically pure LSD was obtained from Lipomed AG, Arlesheim, Switzerland. The same material was used for the pilot study (Gasser et al., 2014) and the studies previously conducted in healthy subjects in Basel (Dolder et al., 2015b; Holze et al., 2020a; Holze et al., 2021; Holze et al., 2020b; Schmid et al., 2015; Strajhar et al., 2016), Zurich (Kraehenmann et al., 2017a; Kraehenmann et al., 2017b; Preller et al., 2017; Preller et al., 2019) and London (Carhart-Harris et al., 2016b; Carhart-Harris et al., 2015; Kaelen et al., 2015). In contrast to the first previous studies, LSD was formulated as drinking solution LSD in dark glass vials (water/alcohol) and not in capsules. This allowed for facilitated regular analytical quality controls and content-uniformity and stability of the formulation throughout the entire study period was documented (Holze et al., 2019) and for the first time for a clinical study in patients using LSD. A Swissmedic approved Good Manufacturing Practice (GMP) facility (Apotheke Dr. C. Hysek) prepared the clinical medication and the placebo (solution without LSD in identical vials) as well as perform the randomization, individualized packaging, labeling and quality control (QC). The production of the investigational medicinal product (IMP) was approved Swissmedic and the use of LSD was authorized by the BAG.

### Randomization and Blinding

Each subject received two treatments. Subjects and investigators were blinded to treatment order. The GMP facility performed the randomization. The treatment order was counterbalanced. A treatment order was assigned to each subject number (code list) and kept by the GMP facility.

### Data Analysis

### Sample Size Estimation

Power analysis was performed with PASS® (Kaysville, Utah). The primary predetermined study endpoint was STAI anxiety. Data from the pilot study was used for the sample size estimation (Gasser et al., 2014; Gasser et al., 2015). In the pilot study, LSD reduced STAI state anxiety scores within-subjects from (mean±SD, [range]) 53.1±13.5 (27-71) to 41.5±9.7 (26-58) by 11.6±9.5 points (raw data available to the PI). Trait anxiety scores decreased from 53.3±11.3 (31-70) to 45.3±10.3 (32-62) by 8.0±7.7 points (Gasser et al., 2014; Gasser et al., 2015). While state or trait scale scores were above 40 in all subjects at screening, scores were below 40 at the baseline measurement before LSD or placebo administration in some subjects. Based on the known pilot study data, a sample size of 6 would achieve 80% power to detect this difference in STAI state anxiety of 11.6 with a known SD of 9.5 and with a significance level (alpha) of 0.05 using a two-sided one-sample t-test. However, these data reflect changes over time (pre-post) without an adequate placebo control and not accounting for the substantial placebo/psychotherapy-associated response (De Candia et al., 2009; Fisher et al., 1999; Laakmann et al., 1998; Lopresti et al., 2014). Assuming a smaller but still clinically relevant (Fisher et al., 1999; Laakmann et al., 1998) reduction in anxiety scores in response to LSD and compared with placebo by 10% with an SD of the change of 15%, a sample size of 18 is needed to achieve 80% power at a significance level of 0.05 using a within-subject comparison. Additionally, we analyzed additional secondary outcomes. Thus, it was planned to include 40 subjects and allow for a maximum of 10 non-replaced drop outs. In the pilot study 70 subjects were interested in participating, 50 did not qualify or declined when briefly evaluated by phone/e-mail and 20 were fully screened to include 12 into the study (Gasser et al., 2014; Gasser et al., 2015). Based on this pilot study data it is expected to fully screen 80 subjects among 240 interested persons to include 40 into the study and end up with at least 30 in the final data analysis.

### Analysis of Outcomes

The data was collected in paper form on the study questionnaires included into the CRFs. Thus, the CRFs include the source data. The data was then entered into the GCP compatible database. STAI and other questionnaire scores were then calculated within the database, using on the respective manuals of the test material. The therapeutic outcomes were analyzed as treated between subjects for the first treatment period as well as within-subjects as LSD versus placebo contrasts. In a first analysis presented here only the patients with generalized anxiety disorder were analyzed because this group was completed while the group with patients with life-threatening illness and anxiety would be analyzed later. Ten patients received LSD first and 11 patients received placebo first and these two groups were compared with each other. The two groups were first compared to document their similarity regarding age, sex distribution, disorder and disorder severity. All outcomes were then compared between LSD and placebo as changes from baseline and separately for each session where the therapeutic effect was assessed (between session visit, and at 2, 8, and 16 weeks after the second treatment session). Contrasts between LSD and placebo were analyzed using T-tests for each time-point and each session and on the difference from baseline, which would correspond to an analysis using baseline as covariate. Then, the LSD and placebo effects were also compared within-subjects. First, differences from baseline before each treatment were calculated for each treatment, outcome measure and time-point. These effects were then compared between LSD and placebo for each outcome and at each time-point. This analysis assessed the effect of LSD compared with placebo within-subjects and in the full cross-over design. Data were analyzed only for those 19 subjects who completed both treatments and not for two subjects included in the between-subject comparison because two subjects only completed the first treatment period. The use of baseline differences accounted for differences in disease severity between and also within-subjects and reduced carry-over effects from the first treatment period. For all these analyses, no corrections for multiple testing were applied. This was a subgroup analysis of the anxiety only patients and not the terminal study analysis of all patients. Statistical analyses will be performed using Statistica® (StatSoft Version 12). Supplementary analyses using mixed effects models or analyses on the scores without accounting for the baseline yielded overall very similar result and are not shown here.

### Protection of Subjects

See LSD-specific toxicity and safety monitoring.

### Risks to the Participants

Physical risks: LSD use is not associated with any known physical risks but it may produce psychiatric complications as described below.

Expected acute adverse effects: Dysphoria, anxiety, mood swings, dream-like state, transient depersonalization and derealization phenomena, mild and transient paranoid thinking, negative experiences (anxiety, dysphoria, bad trips), tremor, restlessness, acute perceptual changes, acutely impaired psychomotor function, mild tachycardia, mild hypertension, nausea, headache, dizziness, trembling, lack or appetite (Johnson et al., 2012; Passie et al., 2008: Schmid et al., 2015; Studerus et al., 2011). No severe or serious adverse effects were expected.

Possible lasting adverse effects: flashback phenomena (see below), psychotic reactions.

Veripunction (screening and EOS): There was a risk for pain, bruising and thrombophlebitis.

Risk to privacy of subjects: Potential risks of data collection include breach of confidentiality. The clinical data is linked to the personal data by a code list kept by the investigators.

Financial risks: There was no risk of expense to the subject besides from traveling costs to the study sites (not covered). Insurance coverage was provided.

### LSD-Specific Toxicity Considerations

The primary safety concerns with hallucinogen research are psychological rather than physiological in nature. Even under unsupervised and unprepared conditions, reactions to hallucinogens involving violence or self-destructive behavior are rare, and it is important not to create an unrealistic account of the dangers of hallucinogens (Johnson et al., 2008). Nonetheless, even infrequent reports of such dangers required that we took seriously such risks and took steps to avoid their occurrence.

Psychological effects: Transient anxiety and depressive reactions were expected in some subjects (Passie et al., 2008; Schmid et al., 2015). In laboratory studies using LSD and other psychoactive substances mild or moderate anticipatory anxiety is common at the beginning of the onset of the drug effect (Griffiths et al., 2006; Liechti et al., 2001: Schmid et al., 2015). These reactions are expected to resolve spontaneously with supportive care by the investigators (Griffiths et al., 2011; Griffiths et al., 2006: Schmid et al., 2015). At the dose of LSD used in the present study, subjects were expected to retain most of their thought control and in contrast to psychotic patients, subjects remained aware of the transient state of the drug-induced experience. Events of more pronounced anxiety, panic attacks or agitation could be treated with benzodiazepine administration if needed. A study psychiatrist was present during the sessions and could be contacted after the sessions. Negative experiences (bad trips) and flashback phenomena may occur, generally in uncontrolled conditions (Strassman, 1984). In the case of any psychiatric complications after the study session and also if the participants wanted to discuss negative experiences in association with the study they could contact the study psychiatrists who offered further assistance beyond the testing days. Self-injurious behavior: people who have taken LSD in uncontrolled settings may engage in reckless behavior, such as driving while intoxicated. This risk was greatly reduced by the continued supervision by the investigators until the effects of the psychoactive substances had completely subsided. Prolonged psychiatric symptoms and/or psychosis after LSD use are rare reactions that were unlikely to occur in the cohort of non-psychotic subjects included in this study. LSD or psilocybin may trigger psychotic episodes in people already vulnerable to psychosis rather than directly causing it. Only non-psychotic and at least 25 year-old subjects were included in this study.

Reproductive and developmental risks: LSD is neither mutagenic nor teratogenic and its chronic use is not associated with birth defects. Pregnant women were excluded from the study and effective birth control was mandatory for female participants and pregnancy tests were done before each test session.

Abuse liability: In Switzerland, LSD is scheduled as a narcotic. LSD possesses little if any abuse liability. Hallucinogens are not self-administered by animals and there is no human LSD dependence syndrome (Passie et al., 2008). Subjects with current substance use were not included in the study but substance use disorder in the past was not an exclusion criterion. Hallucinogens have been used and are being investigated in several substance use disorders including opioid (Belleville et al., 1956; Ross, 2012; Savage et al., 1973), alcohol (Bogenschutz et al., 2015; Krebs et al., 2012; Kurland et al., 1967; Liester. 2014; Ludwig et al., 1969; Mangini, 1998; Pahnke et al., 1970), and nicotine dependence (Johnson et al., 2014). Illicit drug use was monitored during the study using repeated urine drug screens.

Neurotoxicity: LSD is not neurotoxic (Nichols, 2016; Passie et al., 2014; Passie et al., 2008).

Flashbacks: Flashbacks can be defined as episodic and short (seconds or minutes) replications of elements of previous substance-related experiences (Holland et al., 2011: Passie et al., 2014). These experiences can be positive or negative. Such phenomena have been reported after the use of many substances and are also prevalent in non-substance using persons (Holland et al., 2011). Clinically significant flashbacks are also defined as hallucinogen persisting perception disorder. This disorder is considered rare but may occur in patients with anxiety disorders and it typically will have a limited course of months to a year (Halpem et al., 1999; Holland et al., 2011; Passie et al., 2014).

### Safety Monitoring

Guidelines for hallucinogen research: The procedures outlined here are based on guidelines for high-dose hallucinogen research (Fischman et al., 1998; Gouzoulis-Mayfrank et al., 1998; Johnson et al., 2008) and on the experience in the conduct of research with psychoactive substances. The procedures are intended to support the safe administration of psychoactive substances while minimizing potential adverse reactions.

Age of participants: LSD and psilocybin have been studied in recent controlled trials in subjects aged 22-62 years (Carhart-Harris et al., 2016b) and 20-64 years (Bogenschutz et al., 2015; Griffiths et al., 2006), respectively. Young age has been associated with increased negative reactions and anxiety to psilocybin (Studerus et al., 2012). Therefore, subjects younger than 25 years are excluded (Studerus et al., 2012). In contrast, older subjects reported less fear of loss of control in response to psilocybin (Studerus et al., 2012). There is no upper age limit but somatic disease and organ insufficiencies are exclusion criteria. Subjects who take medications that may (negatively) interfere with the study or the substances used are excluded. Medications known to alter the effects of hallucinogens are: tricyclic antidepressants, lithium, serotonin uptake inhibitors, antipsychotics, and monoamine oxidase inhibitors (Johnson et al., 2008).

Other psychiatric disorders: The psychiatric screening criteria are important for minimizing the already low chances of precipitating a longer-term psychotic reaction to LSD. Subjects who had a present or past history of meeting DSM-IV criteria for schizophrenia or other psychotic disorders or due to a medical condition) or bipolar disorder were excluded. The above are the most important conditions to exclude for ensuring safety. Subjects with a first-degree relative with these disorders were also excluded. Other psychiatric disorders in addition to anxiety such as co-morbid depression, obsessive-compulsive disorders, or previous substance use disorder were not excluded because hallucinogens had been used in patients with these disorders or specifically to treat these disorders (Gasser et al., 2015; Grob et al., 2011; Krebs et al., 2012; Moreno et al., 2006: Ross, 2012).

Predictors for response: Important factors that predict more pleasant and mystical-type experiences following hallucinogen administration in a controlled research environment are: high scores of the personality trait of Absorption (open to new experiences) and having experienced few psychological problems in the past weeks before the test sessions (Studerus et al., 2012). Factors associated with an unpleasant and/or anxious reaction to the hallucinogen are: low age, emotional lability, and a setting involving a brain scan (Johnson et al., 2008; Studerus et al., 2012). With regard to personality, subjects who are more open to new experiences including the use of hallucinogens would more likely be interested in participating in the study and this self-selection bias enhances the safety of such research (Johnson et al., 2008; Studerus et al., 2011). Patients with a previous severe adverse reaction to a hallucinogen are not included. The investigator asked subjects whether participants had recently experienced psychological problems that may have a negative impact on the experience before each session and sessions could be postponed or stopped if this was the case.

Drug experience: Previous experience with psychoactive drugs may influence the response to psychoactive substances. In controlled studies with psilocybin, drug use and pre-experience with hallucinogens only moderately affected the psilocybin response (Studerus et al., 2012). Hallucinogen-naïve subjects tended to report overall stronger psilocybin effects (Studerus et al., 2012). Subjects who sometimes smoked cannabis (more than once per month) experienced more pleasurable effects and a trend towards less anxiety compared to subjects who rarely used THC (Studerus et al., 2012). No difference was found in the response to LSD between healthy participants with previous experience with hallucinogen use and hallucinogen-naïve subjects (Schmid et al., 2015). Of note, subjects with regular drug use were not included in the above study (Studerus et al., 2012). The present study also included mostly patients with no or only limited previous drug exposures similar to previous studies using LSD (Gasser et al., 2014: Gasser et al., 2015; Schmid et al., 2015) and similar to studies conducted by others using psilocybin (Griffiths et al., 2011; Griffiths et al., 2006; Studerus et al., 2011).

Study personnel: The interpersonal atmosphere is critical for the response to a hallucinogen (Johnson et al., 2008). The study personnel who was present with the volunteers during the sessions had to be knowledgeable about the potential medical and psychological adverse reactions to the substances (Johnson et al., 2008). The personnel should also have human relation skills and should be familiar with the assessment of altered states of consciousness induced with hallucinogens (Johnson et al., 2008). Clinical sensitivity (e.g. empathy, respect) is considered more important than formal degrees when considering personnel qualifications (Johnson et al., 2008). In the present study, the investigators were experienced in the care for research subjects following treatment with psychoactive substances and were present during the substance effect (up to 12 hours). The volunteer was never alone during the acute substance effects (Johnson et al., 2008). The investigator knew the volunteer from the screening and preparatory visits. The study physician who was present during the session also conducted the screening session with the volunteer to establish a good interpersonal relationship.

### Safety Procedures During the Session

During the session subjects were under constant supervision. One person was always present in the session room with the participant. If the participant needed to use the restroom, he/her were escorted to the rest room. The door was not locked (staff had key). The personnel made sure that no participant could leave the research site during the substance effect. In any unexpected event (fire alarm or other) one person was to stay with the research subject at all times.

Adverse cardiovascular effects: Only mild cardiostimulant effects were expected. Cardiovascular effects (blood pressure and heart rate) were repeatedly measured. Closer monitoring was to be implemented if blood pressure values exceed 180/120 mm Hg or decrease below 90 mm Hg for systolic blood pressure. Treatment of a hypertensive reaction (P_{sys}>220 mmHg) would have included lorazepam and nitroglycerine. Treatment of hypotension would have included Trendelenburg position. Cardiac arrest would have triggered immediate cardiopulmonary reanimation and call to the ambulance.

Headaches: LSD may produce transient headaches (Schmid et al., 2015). In the pilot study, one participant required acetaminophen for a moderate headache the day after an LSD session. Conversely, LSD reportedly reduces episodes of cluster headache and migraine (Davenport. 2016; Karst et al., 2010; Sewell et al., 2006). In the pilot study, LSD markedly reduced the number of migraine attacks in two migraine patients.

Pain: Some patients may have need pain medications during the sessions due to their somatic illness. In the pilot study, three patients received their usual pain medication during the sessions.

Adverse psychological reactions: It was expected that LSD may produce transient dysphoric reactions and controllable apprehension/anxiety despite its overall positive mood effects. Adverse reactions (“bad trips”) were expected to be minimized by the controlled setting, the participant selection criteria and participant preparation described above and the interpersonal support of the participants provided by the personnel. Subjects were constantly and carefully observed by the investigator for signs of psychological distress. Unexpected severe anxiety would first be treated with psychological support by the study psychiatrist followed by administration of a benzodiazepine. Personal support and reassurance is the most appropriate and important response to and untoward psychological reactions. If needed, subjects were to be reassured with a touch to the arm/shoulder and verbally reminded that they are in a research study and had taken psychoactive substances and that they would return to normal consciousness in a few hours. Subjects would in general be advised to accept the extraordinary feelings and to surrender to the experience rather than attempting to talk them down or to distract them from their experience (Johnson et al., 2008). These techniques were expected to be sufficient in almost all cases and had successfully been used by the study team. It is unlikely that medications are needed to control panic in healthy subjects (Hasier et al., 2004; Johnson et al., 2008; Schmid et al., 2015) but this was expected in some of the anxiety disorder patients in this study as in the pilot study in patients with anxiety disorder (Gasser et al., 2014; Gasser et al., 2015). In the pilot study, three patients received benzodiazepines during the study but not during the actual LSD sessions (Gasser et al., 2014).

Dizziness/gait control: Except during the peak drug effect, subjects were able to ambulate without difficulty (Schmid et al., 2015). However, the perceptual and proprioceptive effects of LSD/psilocybin makes walking more difficult and guidance may be helpful.

Other AEs. All other adverse reactions were treated as appropriate and needed based on clinical judgment by the study physician. Nausea or headaches during the session would ideally not be treated with medications until the effects have completely resolved to avoid drug-drug interactions. Paracetamol could be used to treat headaches after the session if needed.

AEs between sessions: These effects were assessed as AE at the beginning of the next session or at the EOS.

Duration of session monitoring: Subjects were closely monitored until the subjective effects have completely ceased. This was expected within 12 hours for LSD. In previous studies using 200 μg of LSD, the effects lasted up to 12 hours (Holze et al., 2021; Schmid et al., 2015). No close monitoring is needed beyond this time and subjects could return home. Thus, twelve hours after LSD/placebo administration participants were allowed to return to their home but only with company and supervision provided by the partner, a relative, or a friend. After the test session subjects were allowed to leave only if the subjective effects had ceased as assessed by the investigator. If supervision was not available or if effects persisted, the night had to be spent at the research site. In this case, the investigator was to be present at the research site but in another room as suggested in the safety guidelines (Johnson et al., 2008).

Post-session safety procedures: Based on previous study experience (Gasser et al., 2014; Gasser et al., 2015; Schmid et al., 2015) no formal follow-up support was needed. Any AE between sessions were to be recorded at the next study visit and at the EOS visit. Earlier meetings were scheduled if needed. Subjects were prohibited to drive a car or to operate any machines within 24 hours of substance administration.

### Monitoring of Toxicity

### Safety Definitions

### Adverse Event (AE):

Any untoward medical occurrence in a clinical trial subject administered an IMP and which does not necessarily have a causal relationship with this treatment. An AE can therefore be any unfavorable and unintended sign (including an abnormal laboratory finding), symptom, or disease temporally associated with the use of an (IMP), whether or not considered related to the IMP.

### Adverse Reaction (AR):

All untoward and unintended responses to an IMP judged by investigator/sponsor as having a reasonable causal relationship to the IMP. The expression reasonable causal relationship means to convey in general that there is evidence or argument to suggest a causal relationship.

### Unexpected Adverse Reaction (UAR):

An AR, the nature or severity of which is not consistent with the applicable product information (e.g. investigators brochure for an unapproved investigational product or summary of product characteristics (SmPC) for an authorized product). When the outcome of the adverse reaction is not consistent with the applicable product information this adverse reaction should be considered as unexpected. Side effects documented in the IB or SmPC which occur in a more severe form than anticipated are also considered as being unexpected.

### Serious Adverse Event (SAE) or Serious Adverse Reaction:

Any untoward medical occurrence or effect that at any dose results in death, is life-threatening, requires hospitalization, or prolongation of existing hospitalization, results in persistent or significant disability or incapacity or is a congenital anomaly or birth defect. In this context, the term life threatening refers to an event in which the trial participant was at immediate risk of death at the time of the event: it does not refer to an event, which might have caused death if it were more severe.

### Suspected Unexpected Serious Adverse Reaction (SUSAR):

Any suspected adverse reaction related to an IMP that is both unexpected and serious.

### Causality:

Most adverse events and adverse reactions that occurred in this study, whether they were serious or not, were to be expected treatment-related toxicities due to the medication used in this study. The assignment of the causality was made by the investigator using the definitions in TABLE 1 below.

TABLE 1

| Relationship | Description |
|---|---|
| Unrelated | There is no evidence of any causal relationship. |
| Unlikely | There is little evidence to suggest there is a |
| | causal relationship (e.g. the |
| | event did not occur within a reasonable |
| | time after administration of the |
| | IMP). There is another reasonable explanation |
| | for the event (e.g. the participant's clinical |
| | condition, other concomitant treatment). |
| Possible | There is some evidence to suggest a causal |
| | relationship (e.g. because |
| | the event occurs within a reasonable time |
| | after administration of the |
| | IMP). However, the influence of other factors |
| | may have contributed to |
| | the event (e.g. the participant's clinical condition, |
| | other concomitant treatments). |
| Probable | There is evidence to suggest a causal relationship |
| | and the influence of other factors is unlikely. |
| Definitely | There is clear evidence to suggest a causal |
| | relationship and other |
| | possible contributing factors can be ruled out. |
| Not | There is insufficient or incomplete evidence to |
| assessable | make a clinical judgment of the causal relationship. |

### Adverse Events (AE) Documentation

AEs were described and recorded on the subject's CRF, regardless of the severity or relationship to the IMP. AEs were rated for severity and the potential relationship to the study interventions was evaluated by the investigator according standard criteria. Subjects with AEs were treated appropriately. Abnormal laboratory values not explained by the patients' disease had to be repeated until normal or until the abnormality could be explained and the subject's safety was not at risk.

### Legal Authorizations

LSD is a scheduled substance in Switzerland (Anhang d der BetmV-Swissmedic). Study officials applied to the BAG for permission to use this substance.

### Study Documentation and Record Keeping

The investigator maintained adequate records to enable the conduct of the study to be fully documented. Copies of protocols, identification codes, CRFs, originals of test result reports, drug dispensing logs, correspondence, records of informed consent and other documents pertaining to the conduct of the study will be kept on file for 10 years in the archive of the University Hospital Basel. All forms should be typed or filled out using a blue or black ball-point pen, and must be legible. Errors should be crossed out but not obliterated, the correction inserted, and the change initialed and dated by the investigator or an authorized person. For each subject enrolled a CRF will be completed and signed by the investigator. This also applies to those subjects who fail to complete the study.

### Quality Control and Quality Assurance

### Training of Personnel and SOPs

The study personnel have completed GCP training. The study was performed in accordance with ICH GCP E6 and according the QMS of the CTU of the University Hospital Basel.

### Monitoring

The study was monitored by the CTU Basel.

### Study Results

Patients with Anxiety Disorder without Somatic Illness

Only the data from the patients with anxiety disorder without somatic illness are presented here. Twenty-one patients were started on treatment and completed the first study period up to week 24. Two patients dropped out and nineteen patients completed the entire study.

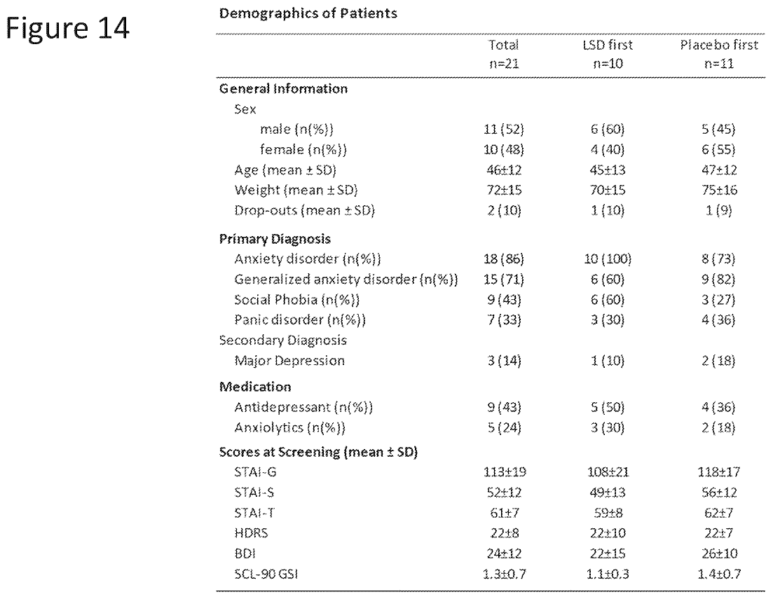

Patient characteristic are shown in FIG. 14. There were 21 patients with anxiety disorder without somatic severe illnesses included in the study (11 men, 10 women). The mean age was 46 years. There were two drop outs after the first study period. Thus, a total of 21 patients were available for the parallel-group comparison and 19 patients could be included in the within-subjects comparison of LSD and placebo.

All patients had a diagnosis of an anxiety disorder and a minimal STAI-S or STAI-T score of 40 at screening. Among the total of 21 patients, there were 18 patients with a primary anxiety disorder, 15 with generalized anxiety disorder, 9 with social phobia, and 7 with panic disorder as their primary diagnosis. Three patients had a diagnosis of major depression. Of all 21 patients 9 had a treatment with an antidepressant (one with lithium) which was tapered off at least 2 weeks before the administration of LSD or placebo in all cases and 5 had an anxiolytic (benzodiazepine). Disease scores were comparable at study inclusion (FIG. 14) and at the baseline treatment.

Patient characteristic were similar in the group receiving LSD first compared with the group receiving placebo first allowing for a valid parallel-group comparison of the LSD and placebo treated patients during the first treatment period (parallel design, between-subjects comparison).

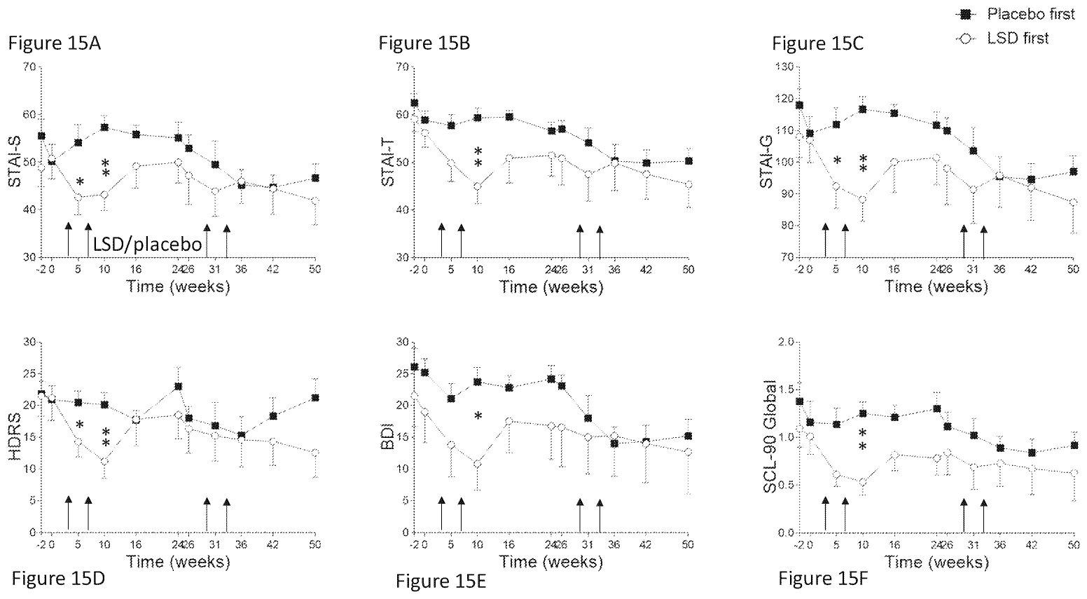

FIGS. 15A-15F show effects of LSD and placebo on ratings of anxiety, depression, and psychological distress. FIG. 15 shows data as mean and SEM values for patients who were treated with placebo first and then LSD (Placebo first, number of patients=11) or patients who were treated with LSD first and then placebo (LSD first, number of patients=10). LSD or placebo were administered at weeks 3 and 8 in the first study period and again at week 29 and 33 in the second study period. LSD significantly reduced STAI-S (FIG. 15A), STAI-T (FIG. 15B), and STAI-G (FIG. 15C) ratings at week 10 (2 weeks after the second dose) compared with placebo (** for p values=0.008, 0.001, and 0.002, respectively). STAI-S and STAI-G ratings were already significantly reduced after the first session (*for p values=0.03 and 0.04, respectively) (FIGS. 15A and 15C). STAI-S, STAI-T, STAI-G ratings continued to be reduced after LSD at weeks 16 and 24 (8 and 16 weeks after the second dose) compared with placebo (FIGS. 15A-C) but did not reach statistical significance. Similarly, LSD significantly reduced scores at week 10 (2 weeks after the second dose) on the HDRS (FIG. 15D), the BDI (FIG. 15E), and the SCL-90 Global (FIG. 15F) compared with placebo (p values were 0.002, 0.02, and 0.004, respectively). On the HDRS, LSD already reduced scores after the first session (*for p=0.04) (FIG. 15D). Effects of LSD on the STAI and SCL-90 Global remained reduced at week 16 and week 24 which was 8 and 16 weeks after the second dose and compared with placebo (FIGS. 15A-C and FIG. 15F) but did not reach statistical significance.

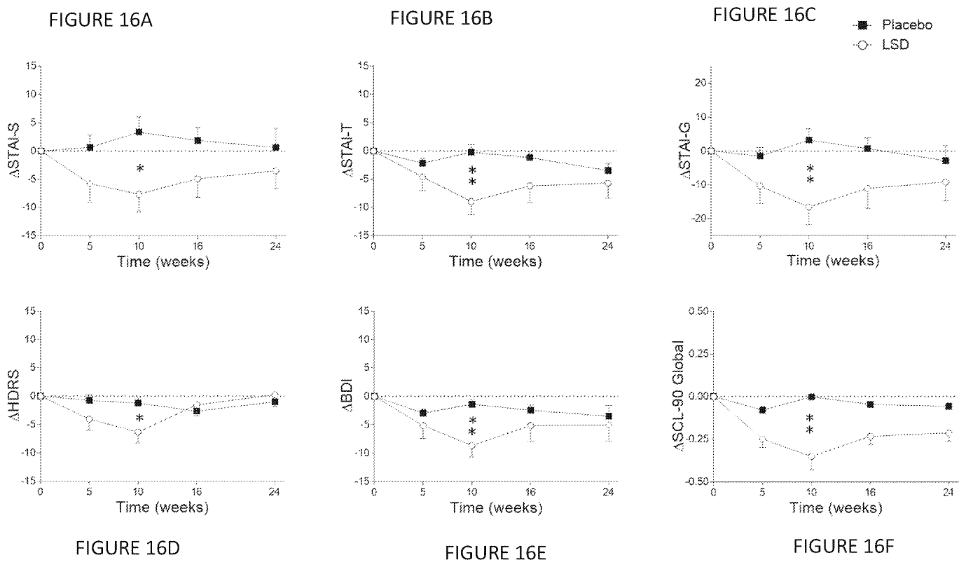

Therapeutic effects were then also evaluated within-subjects making use of the cross-over design of the study where 19 patients received both treatments (one patient received only placebo and one only LSD during the first treatment period). For some measurements and time points data was missing from one or two subjects. As illustrated in FIGS. 15A-15F, there were carry-over effects because the therapeutic effect of LSD in the first treatment phase lasted into the second treatment phase. To account for any shifts in baseline values, data were analyzed as differences from baseline (week 0 or week 26 time points). LSD significantly reduced symptom scores on all measures (STAI-S, STAI-T, STAI-G, HDRS, BDI, and SCL-90 Global) compared with placebo at 2 weeks after the second administration (p values=0.01, 0.003, 0.003, 0.03, 0.002, and 0.004, respectively) compared with placebo (FIGS. 16A-16F).

FIGS. 15A-15F also provide important information on the effect of LSD during the first as compared to its effects during the second treatment period. Effects were clearly present after the first and first two administrations during the first treatment period. Effects were also present during the second treatment period. However, the placebo generally had a similar effect to LSD during the second treatment period in those subjects who already had LSD during the first treatment. This can be explained by a conditioned response in that participant met with the same therapist as during the first session with LSD and in the same setting. This would mean that additional non-drug sessions following an LSD session with the present invention and in patients with anxiety disorder may be beneficial if performed by the same therapist and within the same setting or if a placebo or lower dose of LSD is used due to the presence of effect conditioned by the first LSD administration.

The data was finally evaluated over time for the LSD condition in 19 patients and not using the placebo data. Again LSD reduced scores on all outcome measures significantly at 2 weeks after the second treatment session and compared within-subjects with the baseline measurements for the STAI-S, STAI-T, STAI-G, HDRS, BDI, and SCL-90 Global score (p values=0.006, 0.01, 0.03, 0.02, 0.03, respectively).

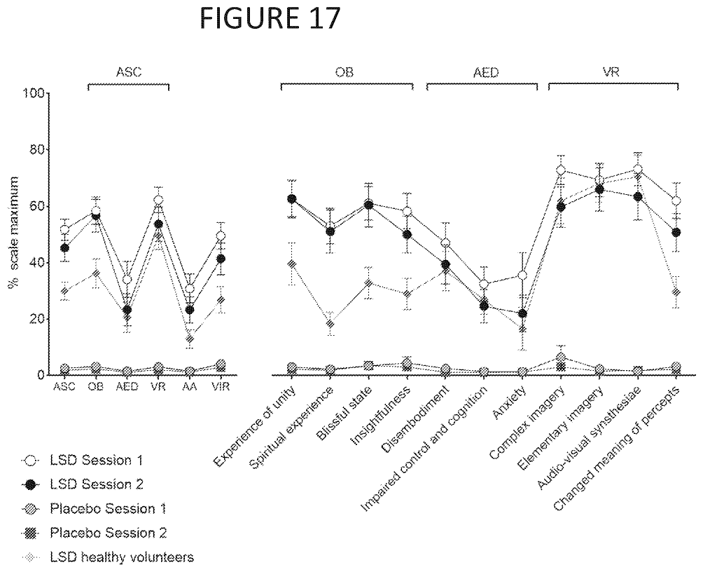

Acute alterations of the mind induced by LSD and placebo are shown in FIG. 17. LSD induced significant and marked alterations in all scales of the 5D-ASC questionnaire (all p<0.001 vs. placebo). Acute LSD effects were comparable at session 1 and 2. Acute effects of LSD were generally greater than those observed in healthy subjects treated in a laboratory setting with the same 200 μg LSD dose (Holze et al., 2021). Specifically, positive effects of LSD (name OB scores) were greater in the anxiety patients compared to healthy subjects while negative effects such as anxiety, impaired control and cognition, and disembodiment as well as the perceptual VR effects were similar. Thus, the invention documents overall positive acute effects of LSD in anxiety patients with an overall similar or better positive versus negative acute effects profile in the patients vs healthy subjects.

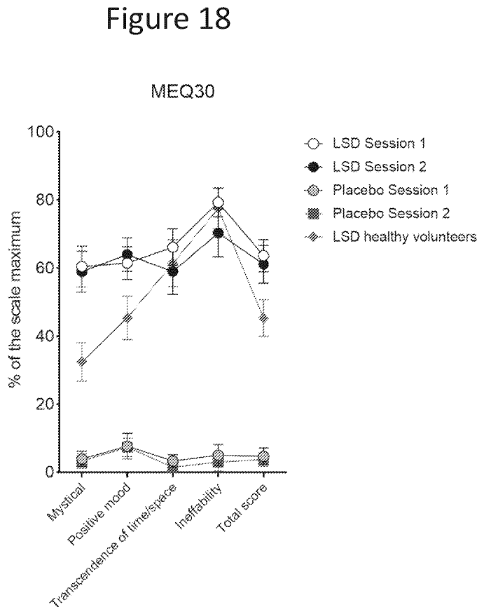

Acute mystical-type effects of LSD and placebo are shown in FIG. 18. LSD significantly and strongly increased ratings of mystical-type experiences on the MEQ30 questionnaire. Effects were similar at the first and second session. Effects tended to be greater in the anxiety patients compared to healthy subjects treated in a laboratory setting with the same 200 μg LSD dose (Holze et al., 2021). In the present invention, LSD clearly produced mystical-type effects known to be associated with positive therapeutic outcomes for psilocybin in other therapeutic studies and patient populations (Garcia-Romeu et al., 2015; Griffiths et al., 2016).

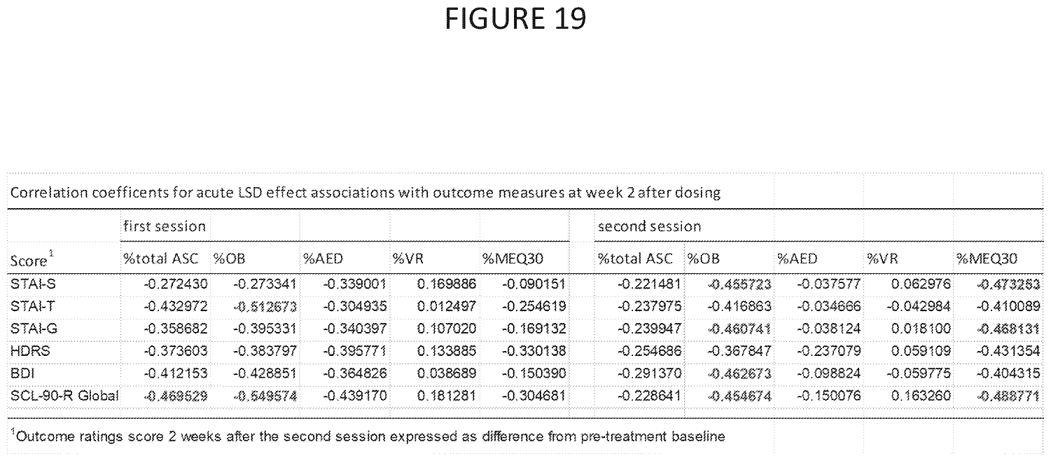

Association of acute effects with long-term therapeutic benefits: Acute effect of LSD on the 5D-ASC and MEQ30 questionnaires were associated with the therapeutic effects of LSD 2 weeks after the second administration. Specifically, % OB scores in particular at the second LSD administration were significantly correlated with therapeutic improvements as evidenced by reductions in STAI-S, STAI-G, BDI, and SCL-90 Global scores (all p values <0.05, Pearson correlations, n=20). Similarly, acute effects of LSD on the MEQ30 at the second LSD session were correlated significantly with reductions in scores on the STAI-S, STAI-G, and SCL-90 Global (all p values <0.05, Pearson correlations, n=20). The correlation coefficients are shown in FIG. 19. The present invention shows that good drug effects of LSD are predictive of good therapeutic outcomes two weeks after treatment. This finding is consistent with studies using psilocybin (Griffiths et al., 2016; Roseman et al., 2017). Additionally, the invention documents that the second session of two sessions best predicted the outcome 2 weeks later. Further, only positive LSD effects as assessed with the OB and MEQ30 scales were predictive while more negative acute effects as assessed with the AED scale were not significantly associated with the therapeutic outcome. Further, visual changes as assessed with the VR score were not predictive of therapeutic response.

### Adverse Events

Adverse events (AEs) specifically asked for during the treatment session included anxiety at onset of the LSD effect in two patients (none with placebo), strong anxiety/paranoia in one patient with LSD (none with placebo), nausea in two patients with LSD (none with placebo) and headache in one patient during LSD (none with placebo).

The paranoia in one patient occurred during the first LSD session and was treated with a benzodiazepine and an antipsychotic and was rated as a serious adverse event (SAE). The dose was then lowered to 100 μg instead of the planned 200 μg in this patients and this treatment was then tolerated well.

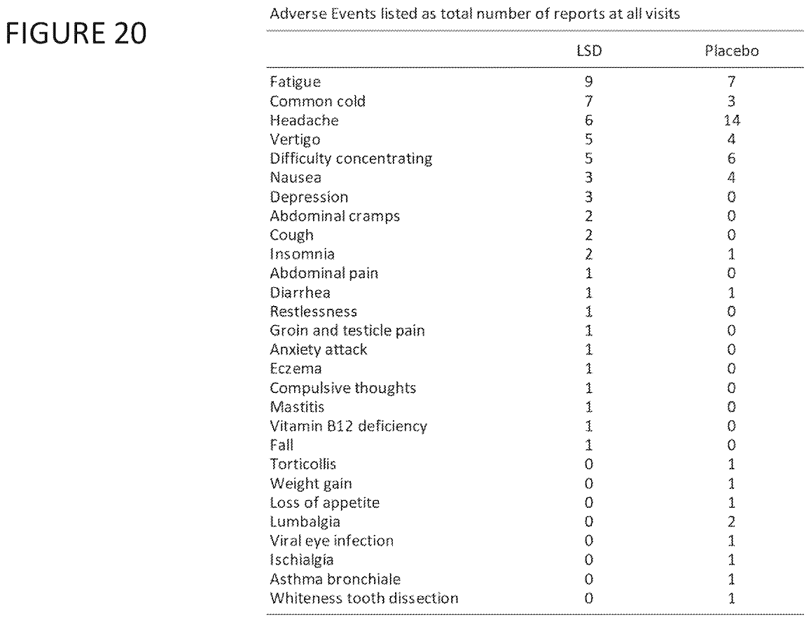

Adverse events (AEs) are listed in a table shown in FIG. 20. Numbers are the total of reported AEs at all visits of a period not including the treatment sessions. The most frequent AEs at these follow-up visit in the absence of substance were: fatigue (LSD 9, placebo 7), common cold (LSD 7, placebo 3), headache (LSD 6, placebo 14), vertigo (LSD 5, placebo 4), difficulty concentrating (LSD 5, placebo 6), nausea (LSD 3, placebo 4), depression (LSD 3, placebo 0).

SAEs in this study included the paranoia noted above during the LSD session in one patient which was an expected reaction to LSD. Another SAE consisted of the hospitalization of one patient due to preexisting obsessive compulsive disorder and during the placebo period which was before the LSD treatment in this patient. Thus this was not a reaction to the substance. Another SAE consisted of a scheduled surgery of nasal septum deviation in one patient during the LSD treatment period and not considered linked to the LSD treatment. Another SAE consisted of a spontaneous abortion in one patient who became pregnant at the end of the LSD treatment phase and neither the pregnancy nor the abortion was considered linked to LSD treatment.

Throughout this application, various publications, including United States patents, are referenced by author and year and patents by number. Full citations for the publications are listed below. The disclosures of these publications and patents in their entireties are hereby incorporated by reference into this application in order to more fully describe the state of the art to which this invention pertains.

The invention has been described in an illustrative manner, and it is to be understood that the terminology, which has been used is intended to be in the nature of words of description rather than of limitation.

Obviously, many modifications and variations of the present invention are possible in light of the above teachings. It is, therefore, to be understood that within the scope of the appended claims, the invention can be practiced otherwise than as specifically described.

### REFERENCES

1. Aghajanian G K, & Bing O H (1964). Persistence of lysergic acid diethylamide in the plasma of human subjects. Clin Pharmacol Ther 5: 611-614.
2. Barrett F S, Johnson M W, & Griffiths R R (2015). Validation of the revised Mystical Experience Questionnaire in experimental sessions with psilocybin. J Psychopharmacol 29:1182-1190.
3. Baumeister D, Barnes G, Giaroli G, & Tracy D (2014). Classical hallucinogens as antidepressants?A review of pharmacodynamics and putative clinical roles. Ther Adv Psychopharmacol 4: 156-169.
4. Beck A T, Ward C H, Mendelson M, Mock J, & Erbaugh J (1961). An inventory for measuring depression. Arch Gen Psychiatry 4: 561-571.
5. Belleville R E, Fraser H F, Isbell H, Wikler A, & Logan C R (1956). Studies on lysergic acid diethylamide (LSD-25): I. Effects in former morphine addicts and development of tolerance during chronic intoxication. AMA Arch Neurol Psychiatry 76: 468-478.
6. Bercel N A, Travis L E, Olinger L B, & Dreikurs E (1956). Model psychoses induced by LSD-25 in normals. I. Psychophysiological investigations, with special reference to the mechanism of the paranoid reaction. AMA Arch Neurol Psychiatry 75: 588-611.
7. Bogenschutz M P (2013). Studying the effects of classic hallucinogens in the treatment of alcoholism: rationale, methodology, and current research with psilocybin. Curr Drug Abuse Rev 6: 17-29.
8. Bogenschutz M P, Forcehimes A A, Pommy J A, Wilcox C E, Barbosa P C, & Strassman R J (2015). Psilocybin-assisted treatment for alcohol dependence: a proof-of-concept study. J Psychopharmacol 29: 289-299.
9. Bonson K R, Buckholtz J W, & Murphy D L (1996a). Chronic administration of serotnergic antidepressants attenuates the subjective effects of LSD in humans. Neuropsychopharmacology 14: 425-436.
10. Bonson K R, & Murphy D L (1996b). Alterations in response to LSD in humans associated with chronic administration of tricyclic antidepressants, monoamine oxidase inhibitors or lithium. Behav Brain Res 73: 229-233.
11. Breitbart W S, & Poppito S R (2014) Individual meaning-centered psychotherapy for patients with advanced cancer: a treatment manual. Oxford University Press: New York.
12. Carhart-Harris R L, Bolstridge M, Day C M J, Rucker J, Watts R, Erritzoe D E, et al. (2018). Psilocybin with psychological support for treatment-resistant depression: six-month follow-up. Psychopharmacology (Ber) 235: 399-408.
13. Carhart-Harris R L, Bolstridge M, Rucker J, Day C M, Erritzoe D, Kaelen M, et al. (2016a). Psilocybin with psychological support for treatment-resistant depression: an open-label feasibility study. Lancet Psychiatry 3: 619-627.
14. Carhart-Harris R L, Kaelen M, Bolstridge M, Williams T M, Williams L T, Underwood R, et al. (2016b). The paradoxical psychological effects of lysergic acid diethylamide (LSD). Psychol Med 46: 1379-1390.
15. Carhart-Harris R L, Kaelen M, Whalley M G, Bolstridge M, Feilding A, & Nutt D J (2015). LSD enhances suggestibility in healthy volunteers. Psychopharmacology (Ber) 232: 785-794.
16. Carhart-Harris R L, Muthukumaraswamy S, Roseman L, Kaelen M, Droog W, Murphy K, et al. (2016c). Neural correlates of the LSD experience revealed by multimodal neuroimaging. Proc Natl Acad Sci USA 113: 4853-4858.
17. Cohen S (1960). Lysergic acid diethylamide: side effects and complications. J Nerv Ment Dis 130: 30-40.
18. Davenport W J (2016). Psychedelic and nonpsychedelic LSD and psilocybin for cluster headache. CMAJ 188: 217.
19. Davis A K, Barrett F S, May D G, Cosimano M P, Sepeda N D, Johnson M W, et al. (2021). Effects of psilocybin-assisted therapy on major depressive disorder: a randomized clinical trial. JAMA Psychiatry 78: 481-489.
20. De Candia M P, Di Sciascio G, Durbano F, Mencacci C, Rubiera M, Aguglia E, et al. (2009). Effects of treatment with etizolam 0.5 mg BID on cognitive performance: a 3-week, multicenter, randomized, double-blind, placebo-controlled, two-treatment, three-period, noninferiority crossover study in patients with anxiety disorder. Clin Ther 31: 2851-2859.
21. Derogatis L R, Rickels K, & Rock A F (1976). The SCL-90 and the MMPI: a step in the validation of a new self-report scale. Br J Psychiatry 128: 280-289.
22. Dittrich A (1998). The standardized psychometric assessment of altered states of consciousness (ASCs) in humans. Pharmacopsychiatry 31 (Suppl 2): 80-84.
23. Dolder P C, Liechti M E, & Rentsch K M (2015a). Development and validation of a rapid turboflow L C-M S/M S method for the quantification of LSD and 2-oxo-3-hydroxy LSD in serum and urine samples of emergency toxicological cases. Anal Bioanal Chem 407: 1577-1584.
24. Dolder P C, Schmid Y, Haschke M, Rentsch K M, & Liechti M E (2015b). Pharmacokinetics and concentration-effect relationship of oral LSD in humans. Int J Neuropsychopharmacol 19: pyv072.
25. Dolder P C, Schmid Y, Mueller F, Borgwardt S, & Liechti M E (2016). LSD acutely impairs fear recognition and enhances emotional empathy and sociality. Neuropsychopharmacology 41: 2638-2646.
26. Dominguez-Clave E, Soler J, Elices M, Pascual J C, Alvarez E, de la Fuente Revenga M, et al. (2016). Ayahuasca: Pharmacology, neuroscience and therapeutic potential. Brain Res Bull 126: 89-101.
27. Dos Santos R G, Osorio F L, Crippa J A, Riba J, Zuardi A W, & Hallak J E (2016). Antidepressive, anxiolytic, and antiaddictive effects of ayahuasca, psilocybin and lysergic acid diethylamide (LSD): a systematic review of clinical trials published in the last 25 years. Ther Adv Psychopharmacol 6: 193-213.
28. Fischman M W, & Johanson C E (1998). Ethical and practical issues involved in behavioral pharmacology research that administers drugs of abuse to human volunteers. Behav Pharmacol 9: 479-498.
29. Fisher P L, & Durham R C (1999). Recovery rates in generalized anxiety disorder following psychological therapy: an analysis of clinically significant change in the STAI-T across outcome studies since 1990. Psychol Med 29: 1425-1434.
30. Franke G H (2002) SCL-90-R: Symptom-Checkliste von L. R. Derogatis: Deutsche Version. Beltz Test GmbH: Göttingen, Germany.
31. Garcia-Romeu A, Davis A K, Erowid F, Erowid E, Griffiths R R, & Johnson M W (2019). Cessation and reduction in alcohol consumption and misuse after psychedelic use. J Psychopharmacol 33: 1088-1101.
32. Garcia-Romeu A, Griffiths R R, & Johnson M W (2015). Psilocybin-occasioned mystical experiences in the treatment of tobacco addiction. Curr Drug Abuse Rev 7: 157-164.
33. Gasser P (1996). Die psycholytische Therapie in der Schweiz von 1988-1993. Schweiz Arch Neurol Psychiatr 147: 59-65.
34. Gasser P, Holstein D, Michel Y, Doblin R, Yazar-Klosinski B, Passie T, et al. (2014). Safety and efficacy of lysergic acid diethylamide-assisted psychotherapy for anxiety associated with life-threatening diseases. J Nerv Ment Dis 202: 513-520.
35. Gasser P, Kirchner K, & Passie T (2015). LSD-assisted psychotherapy for anxiety associated with a life-threatening disease: a qualitative study of acute and sustained subjective effects. J Psychopharmacol 29: 57-68.
36. Gouzoulis-Mayfrank E, Schneider F, Friedrich J, Spitzer M, Thelen B, & Sass H (1998). Methodological issues of human experimental research with hallucinogens. Pharmacopsychiatry 31 Suppl 2: 114-118.
37. Griffiths R, Richards W, Johnson M, McCann U, & Jesse R (2008). Mystical-type experiences occasioned by psilocybin mediate the attribution of personal meaning and spiritual significance 14 months later. J Psychopharmacol 22: 621-632.
38. Griffiths R R, Johnson M W, Carducci M A, Umbricht A, Richards W A, Richards B D, et al. (2016). Psilocybin produces substantial and sustained decreases in depression and anxiety in patients with life-threatening cancer: a randomized double-blind trial. J Psychopharmacol 30: 1181-1197.
39. Griffiths R R, Johnson M W, Richards W A, Richards B D, McCann U, & Jesse R (2011). Psilocybin occasioned mystical-type experiences: immediate and persisting dose-related effects. Psychopharmacology 218: 649-665.
40. Griffiths R R, Richards W A, McCann U, & Jesse R (2006). Psilocybin can occasion mystical-type experiences having substantial and sustained personal meaning and spiritual significance. Psychopharmacology (Ber) 187: 268-283; discussion 284-292.
41. Grob C S, Danforth A L, Chopra G S, Hagerty M, McKay C R, Halberstadt A L, et al. (2011). Pilot study of psilocybin treatment for anxiety in patients with advanced-stage cancer. Arch Gen Psychiatry 68: 71-78.
42. Grof S, Goodman L E, Richards W A, & Kurland A A (1973). LSD-assisted psychotherapy in patients with terminal cancer. Int Pharmacopsychiatry 8: 129-144.
43. Halpem J H, & Pope H G, Jr. (1999). Do hallucinogens cause residual neuropsychological toxicity?Drug Alcohol Depend 53: 247-256.
44. Hamilton M (1960). A rating scale for depression. J Neurol Neurosurg Psychiatry 23: 56-62.
45. Hamilton M (1980). Rating depressive patients. J Clin Psychiatry 41: 21-24.
46. Hasler F, Grimberg U, Benz M A, Huber T, & Vollenweider F X (2004). Acute psychological and physiological effects of psilocybin in healthy humans: a double-blind, placebo-controlled dose-effect study. Psychopharmacology 172: 145-156.
47. Hautzinger M, Keller F, & Kühner C (2006) BDI II: Beck Depressions-Inventar Revision. Harcourt Test Services: Frankfurt/Main, Germany.
48. Hintzen A, & Passie T (2010) The pharmacology of LSD: a critical review. Oxford University Press: Oxford.
49. Hofmann A (1979). How LSD originated. J Psychedelic Drugs 11: 53-60.
50. Holland D, & Passie T (2011) Flaschback-Phänomene. Verlag für Wissenschaft und Bildung: Berlin, Germany.
51. Holze F, Duthaler U, Vizeli P, Muller F, Borgwardt S, & Liechti M E (2019). Pharmacokinetics and subjective effects of a novel oral LSD formulation in healthy subjects. Br J Clin Pharmacol 85:1474-1483.
52. Holze F, Liechti M E, Hutten N, Mason N L, Dolder P C, Theunissen E L, et al. (2020a). Pharmacokinetics and pharmacodynamics of lysergic acid diethylamide microdoses in healthy participants. Clinical pharmacology and therapeutics.
53. Holze F, Vizeli P, Ley L, Muller F, Dolder P, Stocker M, et al. (2021). Acute dose-dependent effects of lysergic acid diethylamide in a double-blind placebo-controlled study in healthy subjects. Neuropsychopharmacology 46: 537-544.
54. Holze F, Vizeli P, Muller F, Ley L, Duerig R, Varghese N, et al. (2020b). Distinct acute effects of LSD, MDMA, and D-amphetamine in healthy subjects. Neuropsychopharmacology 45: 462-471.
55. Hysek C M, Schmid Y, Simmler L D, Domes G, Heinrichs M, Eisenegger C, et al. (2014). MDMA enhances emotional empathy and prosocial behavior. Soc Cogn Affect Neurosci 9: 1645-1652.
56. Janke W, & Debus G (1978) Die Eigenschaftswörterliste. Hogrefe: Göttingen.
57. Johansen P O, & Krebs T S (2015). Psychedelics not linked to mental health problems or suicidal behavior: a population study. J Psychopharmacol 29: 270-279.
58. Johnson M, Richards W, & Griffiths R (2008). Human hallucinogen research: guidelines for safety. J Psychopharmacol 22: 603-620.
59. Johnson M W, Garcia-Romeu A, Cosimano M P, & Griffiths R R (2014). Pilot study of the 5-HT2AR agonist psilocybin in the treatment of tobacco addiction. J Psychopharmacol 28: 983-992.
60. Johnson M W, Garcia-Romeu A, & Griffiths R R (2016). Long-term follow-up of psilocybin-facilitated smoking cessation. Am J Drug Alcohol Abuse 43: 55-60.
61. Johnson M W, Sewell R A, & Griffiths R R (2012). Psilocybin dose-dependently causes delayed, transient headaches in healthy volunteers. Drug Alcohol Depend 123: 132-140.
62. Johnston L D, O'Malley P M, Bachmann J G, Schulenberg J E, & Miech R A (2016). Monitoring the future: national survey results on drug use, 1975-2015: Volume 2. College students and adults ages 19-55 Ann Arbor: Institute for Social Research, University of Michigan.
63. Kaelen M, Barrett F S, Roseman L, Lorenz R, Family N, Bolstridge M, et al. (2015). LSD enhances the emotional response to music. Psychopharmacology (Berl) 232: 3607-3614.
64. Kaelen M, Roseman L, Kahan J, Santos-Ribeiro A, Orban C, Lorenz R, et al. (2016). LSD modulates music-induced imagery via changes in parahippocampal connectivity. Eur Neuropsychopharmacol 26: 1099-1109.
65. Karst M, Halpem J H, Bemateck M, & Passie T (2010). The non-hallucinogen 2-bromo-lysergic acid diethylamide as preventative treatment for duster headache: an open, non-randomized case series. Cephalalgia 30: 1140-1144.
66. Kast E (1966). LSD and the dying patient. Chic Med Sch Q 26: 80-87.
67. Kast E C, & Collins V J (1964). Study Of Lysergic Acid Diethylamide As An Analgesic Agent. Anesth Analg 43: 285-291.
68. Koelle G B (1958). The pharmacology of mescaline and D-lysergic acid diethylamide (LSD). N Engl J Med 258: 25-32.
69. Kometer M, Schmidt A, Bachmann R, Studerus E, Seifritz E, & Vollenweider F X (2012). Psilocybin biases facial recognition, goal-directed behavior, and mood state toward positive relative to negative emotions through different serotonergic subreceptors. Biol Psychiatry 72: 898-906.
70. Kraehenmann R, Pokomy D, Aicher H, Preller K H, Pokomy T, Bosch O G, et al. (2017a). LSD Increases Primary Process Thinking via Serotonin 2A Receptor Activation. Front Pharmacol 8: 814.
71. Kraehenmann R, Pokomy D, Vollenweider L, Preller K H, Pokomy T, Seifritz E, et al. (2017b). Dreamlike effects of LSD on waking imagery in humans depend on serotonin 2A receptor activation. Psychopharmacology (Berl) 234: 2031-2046.
72. Kraehenmann R, Preller K H, Scheidegger M, Pokomy T, Bosch O G, Seifritz E, et al. (2015). Psilocybin-induced decrease in amygdala reactivity correlates with enhanced positive mood in healthy volunteers. Biol Psychiatry 78: 572-581.
73. Krebs T S, & Johansen P O (2012). Lysergic acid diethylamide (LSD) for alcoholism: meta-analysis of randomized controlled trials. J Psychopharmacol 26: 994-1002.
74. Krebs T S, & Johansen P O (2013a). Over 30 million psychedelic users in the United States. F1000 Res 2: 98.
75. Krebs T S, & Johansen P O (2013b). Psychedelics and mental health: a population study. PLoS One 8: e63972.
76. Kuhner C, Burger C, Keller F, & Hautzinger M (2007). [Reliability and validity of the Revised Beck Depression Inventory (BDI-II). Results from German samples]. Nervenarzt 78: 651-656.
77. Kupferschmidt K (2014). High hopes. Science 345: 18-23.
78. Kurland A A, Unger S, Shaffer J W, & Savage C (1967). Psychedelic therapy utilizing LSD in the treatment of the alcoholic patient: a preliminary report. Am J Psychiatry 123: 1202-1209.
79. Laakmann G, Schule C, Lorkowski G, Baghai T, Kuhn K, & Ehrentraut S (1998). Buspirone and lorazepam in the treatment of generalized anxiety disorder in outpatients. Psychopharmacology (Ber) 136: 357-366.
80. Lebedev A V, Kaelen M, Lovden M, Nilsson J, Feilding A, Nutt D J, et al. (2016). LSD-induced entropic brain activity predicts subsequent personality change. Hum Brain Mapp 37: 3203-3213.
81. Leuner H C (1969). [State of development of guided affective imagery]. Z Psychother Med Psychol 19: 177-187.
82. Liechti M E (2017). Modem clinical research on LSD. Neuropsychopharmacology 42: 2114-2127.
83. Liechti M E, Dolder P C, & Schmid Y (2017). Alterations in conciousness and mystical-type experiences after acute LSD in humans. Psychopharmacology 234: 1499-1510.
84. Liechti M E, Gamma A, & Vollenweider F X (2001). Gender differences in the subjective effects of MDMA. Psychopharmacology (Ber) 154:161-168.
85. Liester M B (2014). A review of lysergic acid diethylamide (LSD) in the treatment of addictions: historical perspectives and future prospects. Curr Drug Abuse Rev 7: 146-156.
86. Lopresti A L, Maes M, Maker G L, Hood S D, & Drummond P D (2014). Curcumin for the treatment of major depression: a randomised, double-blind, placebo controlled study. J Affect Disord 167: 368-375.
87. Ludwig A, Levine J, Stark L, & Lazar R (1969). A clinical study of LSD treatment in alcoholism. Am J Psychiatry 126: 59-69.
88. MacLean K A, Johnson M W, & Griffiths R R (2011). Mystical experiences occasioned by the hallucinogen psilocybin lead to increases in the personality domain of openness. J Psychopharmacol 25: 1453-1461.
89. MacLean K A, Leoutsakos J M, Johnson M W, & Griffiths R R (2012). Factor analysis of the Mystical Experience Questionnaire: a study of experiences occasioned by the hallucinogen psilocybin. J Sci Study Relig 51: 721-737.
90. Mangini M (1998). Treatment of alcoholism using psychedelic drugs: a review of the program of research. J Psychoactive Drugs 30: 381-418.
91. Moreno F A, Wiegand C B, Taitano E K, & Delgado P L (2006). Safety, tolerability, and efficacy of psilocybin in 9 patients with obsessive-compulsive disorder. J Clin Psychiatry 67: 1735-1740.
92. Nichols D E (2004). Hallucinogens. Pharmacol Ther 101: 131-181.
93. Nichols D E (2016). Psychedelics. Pharmacol Rev 68: 264-355.
94. Northcote J (2011). Young adults' decision making surrounding heavy drinking: A multi-staged model of planned behaviour. Soc Sci Med 72: 2020-2025.
95. Pahnke W N, Kurland A A, Goodman L E, & Richards W A (1969). LSD-assisted psychotherapy with terminal cancer patients. Curr Psychiatr Ther 9: 144-152.
96. Pahnke W N, Kurland A A, Unger S, Savage C, & Grof S (1970). The experimental use of psychedelic (LSD) psychotherapy. JAMA 212: 1856-1863.
97. Palhano-Fontes F, Barreto D, Onias H, Andrade K C, Novaes M M, Pessoa J A, et al. (2019). Rapid antidepressant effects of the psychedelic ayahuasca in treatment-resistant depression: a randomized placebo-controlled trial. Psychol Med 49: 655-663.
98. Passie T, & Halpem J H (2014). The pharmacology of hallucinogens. In The ASAM principles of addiction medicine. ed Ries R., K. Wolters Kluver Alphen aan de Rijn, The Netherlands, pp 235-255.
99. Passie T, Halpem J H, Stichtenoth D O, Emrich H M, & Hintzen A (2008). The pharmacology of lysergic acid diethylamide: a review. CNS Neurosci Ther 14: 295-314.
100. Preller K H, Herdener M, Pokomy T, Planzer A, Kraehenmann R, Stämpfli P, et al. (2017). The fabric of meaning and subjective effects in LSD-induced states depend on serotonin 2A receptor activation Curr Biol 27: 451-457.
101. Preller K H, Razi A, Zeidman P, Stampfli P, Friston K J, & Vollenweider F X (2019). Effective connectivity changes in LSD-induced altered states of consciousness in humans. Proc Natl Acad Sci USA 116: 2743-2748.
102. Rickli A, Luethi D, Reinisch J, Buchy D, Hoener M C, & Liechti M E (2015). Receptor interaction profiles of novel N-2-methoxybenzyl (NBOMe) derivatives of 2,5-dimethoxy-substituted phenethylamines (2C drugs). Neuropharmacology 99: 546-553.
103. Rickli A, Moning O D, Hoener M C, & Liechti M E (2016). Receptor interaction profiles of novel psychoactive tryptamines compared with classic hallucinogens. Eur Neuropsychopharmacol 26:1327-1337.
104. Roseman L, Nutt D J, & Carhart-Harris R L (2017). Quality of acute psychedelic experience predicts therapeutic efficacy of psilocybin for treatment-resistant depression. Front Pharmacol 8: 974.
105. Roseman L, Sereno M I, Leech R, Kaelen M, Orban C, McGonigle J, et al. (2016). LSD alters eyes-closed functional connectivity within the early visual cortex in a retinotopic fashion. Hum Brain Mapp 37: 3031-3040.
106. Ross S (2012). Serotonergic hallucinogens and emerging targets for addiction pharmacotherapies. Psychiatr Clin North Am 35: 357-374.
107. Ross S, Bossis A, Guss J, Agin-Liebes G, Malone T, Cohen B, et al. (2016). Rapid and sustained symptom reduction following psilocybin treatment for anxiety and depression in patients with life-threatening cancer a randomized controlled trial. J Psychopharmacol 30: 1165-1180.
108. Sanches R F, de Lima Osorio F, Dos Santos R G, Macedo L R, Maia-de-Oliveira J P, Wichert-Ana L, et al. (2016). Antidepressant Effects of a Single Dose of Ayahuasca in Patients With Recurrent Depression: A SPECT Study. J Clin Psychopharmacol 36: 77-81.
109. Savage C (1952). Lysergic acid diethylamide; a clinical-psychological study. Am J Psychiatry 108: 896-900.
110. Savage C, & McCabe O L (1973). Residential psychedelic (LSD) therapy for narcotic addict: a controlled study. Arch Gen Psychiatry 28: 808-814.
111. Schmid Y, Enzler F, Gasser P, Grouzmann E, Preller K H, Vollenweider F X, et al. (2015). Acute effects of lysergic acid diethylamide in healthy subjects. Biol Psychiatry 78: 544-553.
112. Schmid Y, Gasser P, Oehen P, & Liechti M E (2021). Acute subjective effects in LSD- and MDMA-assisted psychotherapy. J Psychopharmacol 35: 362-374.
113. Schmidt A, Kometer M, Bachmann R, Seifritz E, & Vollenweider F (2013). The NMDA antagonist ketamine and the 5-H T agonist psilocybin produce dissociable effects on structural encoding of emotional face expressions. Psychopharmacology (Berl) 225: 227-239.
114. Schmitz N, Hartkamp N, Kiuse J, Franke G H, Reister G, & Tress W (2000). The Symptom Check-List-90-R (SCL-90-R): a German validation study. Qual Life Res 9: 185-193.
115. Sewell R A, Halpern J H, & Pope H G, Jr. (2006). Response of cluster headache to psilocybin and LSD. Neurology 66: 1920-1922.
116. Speth J, Speth C, Kaelen M, Schloerscheidt A M, Feilding A, Nutt D J, et al. (2016). Decreased mental time travel to the past correlates with default-mode network disintegration under lysergic acid diethylamide. J Psychopharmacol 30: 344-353.
117. Spielberger C D, Gorusch R L, Lushene R, Vagg P R, & Jacobs G A (1983). Manual for the state trait anxiety inventory. Consulting Psychologists.
118. Strajhar P, Schmid Y, Liakoni E, Dolder P C, Rentsch K M, Kratschmar D V, et al. (2016). Acute effects of lysergic acid diethylamide on circulating steroid levels in healthy subjects. J Neuroendocrinol 28: 12374.
119. Strassman R J (1984). Adverse reactions to psychodelic drugs: a review of the literature J Nerv Ment Dis 172: 577-595.
120. Strassman R J (1992). Human halluciongen interactions with drugs affecting serotonergic neurotransmission. Neuropsychopharmacology 7: 241-243.
121. Studerus E, Gamma A, Kometer M, & Vollenweider F X (2012). Prediction of psilocybin response in healthy volunteers. PLoS One 7: e30800.
122. Studerus E, Gamma A, & Vollenweider F X (2010). Psychometric evaluation of the altered states of consciousness rating scale (OAV). PLoS One 5: e12412.
123. Studerus E, Kometer M, Hasler F, & Vollenweider F X (2011). Acute, subacute and long-term subjective effects of psilocybin in healthy humans: a pooled analysis of experimental studies. J Psychopharmacol 25: 1434-1452.
124. Tagliazucchi E, Roseman L, Kaelen M, Orban C, Muthukumaraswamy S D, Murphy K, et al. (2016). Increased global functional connectivity correlates with LSD-induced ego dissolution. Curr Biol 26: 1043-1050.
125. Terhune D B, Luke D P, Kaelen M, Bolstridge M, Feilding A, Nutt D, et al. (2016). A placebo-controlled investigation of synaesthesia-like experiences under LSD. Neuropsychologia 88: 28-34.
126. Vollenweider F X, & Preller K H (2020). Psychedelic drugs: neurobiology and potential for treatment of psychiatric disorders. Nat Rev Neurosci 21: 611-624.
127. Vollenweider F X, Vollenweider-Scherpenhuyzen M F, Babler A, Vogel H, & Hell D (1998). Psilocybin induces schizophrenia-like psychosis in humans via a serotonin-2 agonist action. Neuroreport 9: 3897-3902.
128. White T L, Lott D C, & de Wit H (2006). Personality and the subjective effects of acute amphetamine in healthy volunteers. Neuropsychopharmacology 31: 1064-1074.
129. Wittchen H U, Wunderlich U, Gruschwitz S, & Zaudig M (1997) SKID-I: Strukturiertes Klinisches Interview für DSM-IV. Hogrefe-Verlag: Göttingen.

1.-17. (canceled)

18. A method of treating generalized anxiety disorder in an individual comprising:
administering 25-400 μg of LSD, or a salt thereof, wherein the administration of 25-400 μg of LSD, or a salt thereof, results in a reduced STAI global score.

19. The method of claim 18, wherein a second dose of LSD, or a salt thereof, is administered four to five weeks after said administering step.

20. The method of claim 18, when the subject also has depression or low mood associated or co-present with the anxiety.

21. The method of claim 18, wherein the administration of LSD, or a salt thereof, results in reduced psychological distress.

22. The method of claim 18, wherein the administration of LSD, or a salt thereof, results in an increased quality of life in the individual.

23. The method of claim 18, further including the step of providing psychotherapy to the individual on separate days before and after administration of LSD, or a salt thereof.

24. The method of claim 23, wherein the administration of LSD, or a salt thereof, enhances the therapeutic effect of the psychotherapy.

25. The method of claim 18, wherein the LSD, or a salt thereof, is administered in the presence of a therapist for 12 hours in a quiet room.

26. The method of claim 25, wherein the individual is lying in a bed or sitting in a chair and listening to music.

27. The method of claim 19, wherein the second dose of LSD, or a salt thereof, is administered in the presence of a therapist for 12 hours in a quiet room.

28. The method of claim 27, wherein the individual is lying in a bed or sitting in a chair and listening to music.

29. The method of claim 18, wherein the individual was tapered off an antidepressant at least two weeks before administration of LSD, or a salt thereof.

30. The method of claim 18, wherein the STAI global score is reduced by 10-points in about four weeks or greater after administration of LSD, or a salt thereof.

31. The method of claim 18, wherein the STAI global score is reduced by 10-points in about ten weeks or greater after administration of LSD, or a salt thereof.

32. The method of claim 18, wherein the STAI global score is reduced by 20-points in about ten weeks or greater after administration of LSD, or a salt thereof.
